(19) United States
(12) Patent Application Publication
(10) Pub. No.: US 2013/0022569 A1
(43) Pub. Date: Jan. 24, 2013

(54) HYDROGELS

(71) Applicants: Kathryn E. Uhrich, New Brunswick, NJ (US); Ashley L. Howell, New Brunswick, NJ (US); Renata Fogaça da Silva, Paulo (BR); Luiz Henrique Catalina, Sao Paulo (BR); Michelle Aimeé Ouimet, Brunswick, NJ (US)

(72) Inventors: Kathryn E. Uhrich, New Brunswick, NJ (US); Ashley L. Howell, New Brunswick, NJ (US); Renata Fogaça da Silva, Paulo (BR); Luiz Henrique Catalina, Sao Paulo (BR); Michelle Aimeé Ouimet, Brunswick, NJ (US)

(21) Appl. No.: 13/473,383

(22) Filed: May 16, 2012

Related U.S. Application Data
(60) Provisional application No. 61/486,647, filed on May 16, 2011.

(51) Int. Cl. A61K 31/79; A61P 17/00; A61P 17/02

(52) U.S. Cl. 424/78.3

(57) ABSTRACT

The invention provides hydrogels comprising bioactive-based poly(anhydride-ester)s (PAEs) and methods for use thereof.

### PRIORITY OF INVENTION

This application claims priority from U.S. Provisional Application No. 61/486,647, filed on May 16, 2011, which application is herein incorporated by reference.

### GOVERNMENT FUNDING

The invention described herein was made with government support under Grant Number NIH-RO1-DE13209 awarded by the National Institutes of Health. The United States Government has certain rights in the invention.

### BACKGROUND OF THE INVENTION

Hydrogels are three-dimensional hydrophilic crosslinked polymer networks that can absorb large volumes of water and biological fluids without dissolving. Hydrogels may be comprised of polymers that are insoluble due to the presence of physical crosslinks (e.g., crystalline regions, intermolecular interactions and entanglements) or chemical crosslinks (e.g., covalent bonding). Poly(N-vinyl-2-pyrrolidone) (PVP) is a common polymer that has been used to produce hydrogels. These systems are becoming increasingly important for a variety of biomedical applications, including drug delivery and topical applications, because they are biocompatible, act as a physical barrier against infection, and have properties similar to that of natural living tissue due to their high water content and soft consistency. To improve healing, bioactives can be chemically and physically incorporated into hydrogel networks. However, hydrogels are limited as drug delivery systems because high drug loading (particularly hydrophobic drugs) is difficult to achieve (Hoare, et al., Polymer 2008, 49, (8), 1993-2007; Costache, et al., Biomaterials 2010, 31, (24), 6336-6343). Moreover, the high water content and large pore size of most hydrogels often result in relatively rapid drug release (hours to days). Ideally, the drug release profile should deliver nearly instantaneous dosage of drug followed by a sustained drug release to maintain the proper concentration of drug at therapeutic levels without causing toxicity issues. Accordingly, there is a need for a hydrogel drug delivery system that allows high loadings and controlled, sustained release of bioactives.

### SUMMARY OF THE INVENTION

Certain embodiments of the present invention provide hydrogels comprising bioactive-based poly(anhydride-ester)s (PAEs), which can be used for controlled, sustained delivery of bioactives.

Certain embodiment of the invention provide a hydrogel comprising (a) a poly(anhydride-ester) comprising a polymer backbone and having a group in the polymer backbone that will yield a bioactive molecule upon hydrolysis of the polymer backbone; and (b) a hydrophilic polymer that is crosslinked with the poly(anhydride-ester).

Certain embodiments provide a method of making a hydrogel as described herein, comprising solvent casting (a) a poly(anhydride-ester) comprising a polymer backbone and having a group in the polymer backbone that will yield a bioactive molecule upon hydrolysis of the polymer backbone; and (b) a hydrophilic polymer; under conditions to provide a hydrogel.

Certain embodiments provide a method of making a hydrogel as described herein, comprising electrospinning (a) a poly(anhydride-ester) comprising a polymer backbone and having a group in the polymer backbone that will yield a bioactive molecule upon hydrolysis of the polymer backbone; and (b) a hydrophilic polymer; under conditions to provide a hydrogel.

Certain embodiments provide a method for promoting wound healing in a mammal, comprising contacting a hydrogel as described herein with a wound of the mammal.

Certain embodiments provide a method of therapeutically treating the skin of a mammal, comprising contacting a hydrogel as described herein with the skin of the mammal.

Certain embodiments provide a poly(anhydride-ester) comprising (a) a polymer backbone and having a group in the polymer backbone that will yield a bioactive molecule upon hydrolysis of the polymer backbone; and (b) one or more linker molecules in the polymer backbone comprising one or more photoreactive double bonds.

### BRIEF DESCRIPTION OF THE FIGURES

FIG. 1. Methodology for generating the hydrogels.

FIG. 2. Swelling ratio (Q) comparison between all PVP/polyanhydride 7:3 hydrogels after 24 hours.

FIG. 3. Swelling ratio (Q) of the blended hydrogels after 24 hours.

FIG. 4. FTIR spectra of PVP/Polyanhydride (7:3) blends PVP (a) PVP/FA (b) PVP/SinA (c) PVP/p-CA (d) PVP/SAA (e).

FIG. 5. SEM images of bioactive PVP-based porous hydrogels with PVP/FA (a), PVP/SinA (b), PVP/p-CA (c), PVP/SAA (d), and (e) PVP.

FIG. 6. Cumulative release profiles of 6 and 0, 1, 5, and 10% (w/w) admixtures of 4, 5, and 4:5 (a-j) over 30 days (a) and expanded image of the first 5 days (b).

FIG. 7. Glass transition temperatures for all admixtures.

FIG. 8. Static contact angle measurements for 1, 5, and 10% (w/w) to determine the influence of admixtures on the polymer surface energy/hydrophobicity.

FIG. 9. Formulation of PVP:SA(diglycolic)PAE hydrogels and methodology for generating these hydrogels.

FIG. 10. Normalized cumulative SA release (%) from PVP:SA(diglycolic) PAE hydrogels with ratios of 60:40 and 50:50. Swelling values (Q) for these two hydrogels are also shown.

FIG. 11. Normalized cumulative SA release (%) from PVP:SA(adipic)PAE hydrogels with ratios of 70:30, 60:40 and 50:50. Swelling values (Q) for these hydrogels are also shown.

FIG. 12. Normalized cumulative SA release (%) from control hydrogels, PVP:poly(anhydride-ether) blends with physically incorporated SA, having ratios of 70:30, 60:40 and 50:50.

FIG. 13. Cytotoxicity studies with L929 mouse fibroblast cell line. Images of cell morphology after exposure to 50:50 PVP:SA(adipic)PAE, 50:50 PVP:SA(diglycolic)PAE and PVP for 48 hours.

FIG. 14. MTS assay to measure cell viability over three days (time points at 24, 48 and 72 hours). PVP:SA(diglycolic)PAE and PVP:SA(adipic)PAE hydrogels with ratios of 70:30, 60:40 and 50:50 were evaluated. PVP also and DMSO were used as controls.

### DETAILED DESCRIPTION

Certain embodiments of the present invention provide hydrogels comprising bioactive-based poly(anhydride-ester)s (PAEs), which can be used for controlled, sustained delivery of bioactives.

Certain embodiments of the present invention provide a hydrogel comprising (a) a poly(anhydride-ester) comprising a polymer backbone and having a group in the polymer backbone that will yield a bioactive molecule upon hydrolysis of the polymer backbone; and (b) a hydrophilic polymer that is crosslinked with the poly(anhydride-ester).

In certain embodiments the bioactive molecule is an antimicrobial, anti-inflammatory, antioxidant or analgesic.

In certain embodiments, the bioactive molecule is an antimicrobial.

In certain embodiments, the antimicrobial is 2-p-sulfanilyanilinoethanol, 4,4′-sulfinyldianiline, 4-sulfanilamidosalicylic acid, acediasulfone, acetosulfone, amikacin, amoxicillin, amphotericin B, ampicillin, apalcillin, apicycline, apramycin, arbekacin, aspoxicillin, azidamfenicol, azithromycin, aztreonam, bacitracin, bambermycin(s), biapenem, brodimoprim, butirosin, capreomycin, carbenicillin, carbomycin, carumonam, cefadroxil, cefamandole, cefatrizine, cefbuperazone, cefclidin, cefdinir, cefditoren, cefepime, cefetamet, cefixime, cefmenoxime, cefininox, cefodizime, cefonicid, cefoperazone, ceforanide, cefotaxime, cefotetan, cefotiam, cefozopran, cefpimizole, cefpiramide, cefpirome, cefprozil, cefroxadine, ceftazidime, cefteram, ceftibuten, ceftriaxone, cefuzonam, cephalexin, cephaloglycin, cephalosporin C, cephradine, chloramphenicol, chlortetracycline, ciprofloxacin, clarithromycin, clinafloxacin, clindamycin, clomocycline, colistin, cyclacillin, dapsone, demeclocycline, diathymosulfone, dibekacin, dihydrostreptomycin, dirithromycin, doxycycline, enoxacin, enviomycin, epicillin, erythromycin, flomoxef, fortimicin(s), gentamicin(s), glucosulfone solasulfone , gramicidin S, gramicidin(s), grepafloxacin, guamecycline, hetacillin, imipenem, isepamicin, josamycin, kanamycin(s), leucomycin(s), lincomycin, lomefloxacin, lucensomycin, lymecycline, meclocycline, meropenem, methacycline, micronomicin, midecamycin(s), minocycline, moxalactam, mupirocin, nadifloxacin, natamycin, neomycin, netilmicin, norfloxacin, oleandomycin, oxytetracycline, p-sulfanilylbenzylamine, panipenem, paromomycin, pazufloxacin, penicillin N, pipacycline, pipemidic acid, polymyxin, primycin, quinacillin, ribostamycin, rifamide, rifampin, rifamycin SV, rifapentine, rifaximin, ristocetin, ritipenem, rokitamycin, rolitetracycline, rosaramycin, roxithromycin, salazosulfadimidine, sancycline, sisomicin, sparfloxacin, spectinomycin, spiramycin, streptomycin, succisulfone, sulfachrysoidine, sulfaloxic acid, sulfamidochrysoidine, sulfanilic acid, sulfoxone, teicoplanin, temafloxacin, temocillin, tetracycline, tetroxoprim, thiamphenicol, thiazolsulfone, thiostrepton, ticarcillin, tigemonam, tobramycin, tosufloxacin, trimethoprim, trospectomycin, trovafloxacin, tuberactinomycin or vancomycin.

In certain embodiments, the bioactive molecule is an anti-inflammatory.

In certain embodiments, the anti-inflammatory is 3-amino-4-hydroxybutyric acid, aceclofenac, alminoprofen, amfenac, bromfenac, bromosaligenin, bumadizon, carprofen, diclofenac, diflunisal, ditazol, enfenamic acid, etodolac, etofenamate, fendosal, fepradinol, flufenamic acid, gentisic acid, glucamethacin, glycol salicylate, meclofenamic acid, mefenamic acid, mesalamine, niflumic acid, olsalazine, oxaceprol, S-adenosylmethionine, salicylic acid, salsalate, sulfasalazine or tolfenamic acid.

In certain embodiments, the bioactive molecule is an antioxidant.

In certain embodiments, the antioxidant is ferulic acid, sinapic acid, or coumaric acid (e.g., p-coumaric acid).

In certain embodiments, the bioactive molecule is an analgesic.

In certain embodiments, the analgesic is oxymorphone, buprenorphine, butorphanol, nalbuphine, butethamine, fenalcomine, hydroxytetracaine, naepaine, orthocaine, piridocaine or salicyl alcohol.

In certain embodiments, the bioactive molecule is a hydroxycinnamate or a salicylate.

In certain embodiments, the bioactive molecule is a hydroxycinnamate.

In certain embodiments, the hydroxycinnamate is selected from ferulic acid, sinapic acid and coumaric acid (e.g., p-coumaric acid). In certain embodiments the hydroxycinnamate is ferulic acid. In certain embodiments the hydroxycinnamate is sinapic acid. In certain embodiments the hydroxycinnamate is p-coumaric acid.

In certain embodiments, the bioactive molecule is a salicylate.

In certain embodiments, the salicylate is salicylic acid.

In certain embodiments, the poly(anhydride-ester) further comprises one or more linker molecules in the polymer backbone.

In certain embodiments, the poly(anhydride-ester) comprises one or more units of formula (I) in the backbone:

—C(═O)X¹-L-X¹C(═O)—O— (I)
wherein
   each X¹ is independently a group that will provide a biologically active compound upon hydrolysis of the polymer; and
   L is independently a linker molecule.

Changes in the bioactive-based polyanhydride backbone may be made, such as changing the linker type to produce a more hydrophilic or hydrophobic polymer. For example, a more hydrophilic linker may be utilized instead of a hydrophobic linker, to produce blended hydrogels with a faster release of the bioactive molecules.

Accordingly, in certain embodiments, each linker molecule is selected from a branched aliphatic, linear aliphatic, and oxygen-containing linker molecule.

In certain embodiments, the branched aliphatic, linear aliphatic, or oxygen-containing linker molecule is C₁-C₁₅.

In certain embodiments, the branched aliphatic, linear aliphatic, or oxygen-containing linker molecule is C₁-C₆.

In certain embodiments, each linker molecule is a branched aliphatic linker molecule.

In certain embodiments, the branched aliphatic linker molecule is derivable from diethylmalonyl chlorides.

In certain embodiments, each linker molecule is a linear aliphatic linker molecule.

In certain embodiments, the linear aliphatic linker molecule is derivable from adipoyl chlorides.

In certain embodiments, each linker molecule is an oxygen-containing linker molecule.

In certain embodiments, the oxygen-containing linker molecule is derivable from diglycolyl chlorides.

In certain embodiments, each linker molecule comprises a photoreactive double bond.

In certain embodiments, the linker molecule comprising a photoreactive double bond is C₁-C₁₅.

In certain embodiments, the linker molecule comprising a photoreactive double bond is C₁-C₆.

In certain embodiments, the linker molecule (e. g. , comprising a photoreactive double bond) is derivable from itaconyl chloride or fumaryl chloride.

In certain embodiments, L is

A-l′-A

wherein A is independently an ester linkage; and each L′ is independently a linker molecule. As used herein, the term ester linkage means —OC(═O)— or —C(═O)O—.

In certain embodiments, L′ is a divalent, branched or unbranched, saturated or unsaturated, hydrocarbon chain, having from 1 to 25 carbon atoms, wherein one or more (e.g. 1, 2, 3, or 4) of the carbon atoms is optionally replaced by (—O—) or (—NR—), and wherein the chain is optionally substituted on carbon with one or more (e.g. 1, 2, 3, or 4) substituents selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy.

In certain embodiments, L′ is a divalent, branched or unbranched, saturated or unsaturated, hydrocarbon chain, having from 1 to 25 carbon atoms, wherein the chain is optionally substituted on carbon with one or more (e.g. 1, 2, 3, or 4) substituents selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy.

In certain embodiments, L′ is a peptide. As used herein, the term “peptide” describes a sequence of 2 to 35 amino acids (e.g. as defined hereinabove) or peptidyl residues. The sequence may be linear or cyclic. For example, a cyclic peptide can be prepared or may result from the formation of disulfide bridges between two cysteine residues in a sequence. Preferably a peptide comprises 3 to 20, or 5 to 15 amino acids. Peptide derivatives can be prepared as disclosed in U.S. Pat. Nos. 4,612,302; 4,853,371; and 4,684,620, or as described in the Examples hereinbelow. Peptide sequences specifically recited herein are written with the amino terminus on the left and the carboxy terminus on the right.

In certain embodiments, L′ is an amino acid. As used herein, the term “amino acid,” comprises the residues of the natural amino acids (e.g. Ala, Arg, Asn, Asp, Cys, Glu, Gln, Gly, His, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆)alkyl, phenyl or benzyl ester or amide; or as an α-methylbenzyl amide). Other suitable amino and carboxy protecting groups are known to those skilled in the art (See for example, Greene, T. W.; Wutz, P. G. M. “Protecting Groups In Organic Synthesis” second edition, 1991, New York, John Wiley & sons, Inc., and references cited therein).

In certain embodiments, L′ is a divalent, branched or unbranched, saturated or unsaturated, hydrocarbon chain, having from 1 to 25 carbon atoms, wherein one or more (e.g. 1, 2, 3, or 4) of the carbon atoms is optionally replaced by (—O—) or (—NR—).

In certain embodiments, L′ is a divalent, branched or unbranched, saturated or unsaturated, hydrocarbon chain, having from 3 to 15 carbon atoms, wherein one or more (e.g. 1, 2, 3, or 4) of the carbon atoms is optionally replaced by (—O—) or (—NR—), and wherein the chain is optionally substituted on carbon with one or more (e.g. 1, 2, 3, or 4) substituents selected from the group consisting of (C₁-C₆)alkoxy, (C₃-C₆)cycloalkyl, (C₁-C₆)alkanoyl, (C₁-C₆)alkanoyloxy, (C₁-C₆)alkoxycarbonyl, (C₁-C₆)alkylthio, azido, cyano, nitro, halo, hydroxy, oxo, carboxy, aryl, aryloxy, heteroaryl, and heteroaryloxy.

In certain embodiments, L′ is a divalent, branched or unbranched, saturated or unsaturated, hydrocarbon chain, having from 3 to 15 carbon atoms, wherein one or more (e.g. 1, 2, 3, or 4) of the carbon atoms is optionally replaced by (—O—) or (—NR—).

In certain embodiments, L′ is a divalent, branched or unbranched, saturated or unsaturated, hydrocarbon chain, having from 3 to 15 carbon atoms.

In certain embodiments, L′ is a divalent, branched or unbranched, hydrocarbon chain, having from 3 to 15 carbon atoms.

In certain embodiments, L′ is a divalent, branched or unbranched, hydrocarbon chain, having from 6 to 10 carbon atoms.

In certain embodiments, L′ is a divalent hydrocarbon chain having 7, 8, or 9 carbon atoms.

In certain embodiments, L′ is a divalent hydrocarbon chain having 8 carbon atoms.

In certain embodiments, L′ is adipic (—CH₂CH₂CH₂CH₂—).

In certain embodiments, L′ is diglycolic (—CH₂OCH₂—).

In certain embodiments, L′ is diethylmalonic (CH₂C(Et)₂CH₂—).

In certain embodiments, the poly(anhydride-ester) is:

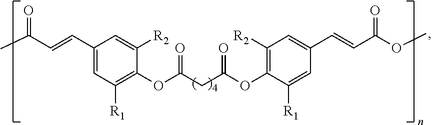

wherein R₁ is OCH₃ and R₂ is H; R₁ is OCH₃ and R₂ is OCH₃; or R₁ is H and R₂ is H; and wherein n is 2 or more.

In certain embodiments, n is 5 or more. In certain embodiments, n is less than 2,000. In certain embodiments, n is less than 1,000. In certain embodiments, n is less than 500. In certain embodiments, n is less than 250. In certain embodiments, n is less than 150. In certain embodiments, n is less than 100. In certain embodiments, n is from about 5 to about 100. In certain embodiments, n is from about 5 to about 90. In certain embodiments, n is from about 5 to about 80. In certain embodiments, n is from about 5 to about 70. In certain embodiments, n is from about 5 to about 60. In certain embodiments, n is from about 10 to about 60. In certain embodiments, n is from about 20 to about 60.

In certain embodiments, the poly(anhydride-ester) is:

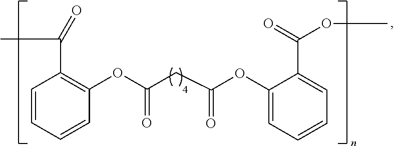

wherein n is 2 or more. In certain embodiments, n is 5 or more. In certain embodiments, n is less than 2,000. In certain embodiments, n is less than 1,000. In certain embodiments, n is less than 500. In certain embodiments, n is less than 250. In certain embodiments, n is less than 150. In certain embodiments, n is less than 100. In certain embodiments, n is from about 5 to about 100. In certain embodiments, n is from about 5 to about 90. In certain embodiments, n is from about 5 to about 80. In certain embodiments, n is from about 5 to about 70. In certain embodiments, n is from about 5 to about 60. In certain embodiments, n is from about 10 to about 60. In certain embodiments, n is from about 20 to about 60.

In certain embodiments, the poly(anhydride-ester) is:

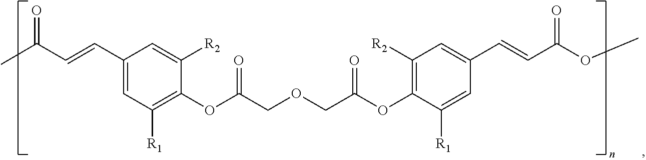

wherein R₁ is OCH₃ and R₂ is H; R₁ is OCH₃ and R₂ is OCH₃; or R₁ is H and R₂ is H; and wherein n is 2 or more. In certain embodiments, n is 5 or more. In certain embodiments, n is less than 2,000. In certain embodiments, n is less than 1,000. In certain embodiments, n is less than 500. In certain embodiments, n is less than 250. In certain embodiments, n is less than 150. In certain embodiments, n is less than 100. In certain embodiments, n is from about 5 to about 100. In certain embodiments, n is from about 5 to about 90. In certain embodiments, n is from about 5 to about 80. In certain embodiments, n is from about 5 to about 70. In certain embodiments, n is from about 5 to about 60. In certain embodiments, n is from about 10 to about 60. In certain embodiments, n is from about 20 to about 60.

In certain embodiments, the poly(anhydride-ester) is:

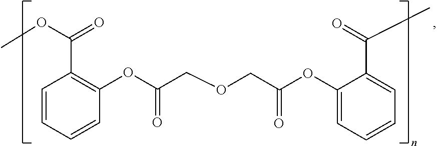

wherein n is 2 or more. In certain embodiments, n is 5 or more. In certain embodiments, n is less than 2,000. In certain embodiments, n is less than 1,000. In certain embodiments, n is less than 500. In certain embodiments, n is less than 250. In certain embodiments, n is less than 150. In certain embodiments, n is less than 100. In certain embodiments, n is from about 5 to about 100. In certain embodiments, n is from about 5 to about 90. In certain embodiments, n is from about 5 to about 80. In certain embodiments, n is from about 5 to about 70. In certain embodiments, n is from about 5 to about 60. In certain embodiments, n is from about 10 to about 60. In certain embodiments, n is from about 20 to about 60.

In certain embodiments, the poly(anhydride-ester) as described herein and prepared in accordance with the present invention has an average molecular weight of about 1,000 daltons to about 100,000 daltons. In certain embodiments, the polymer has an average molecular weight of about 5,000 daltons to about 100,000 daltons. In certain embodiments, the polymer has an average molecular weight of about 5,000 daltons to about 50,000 daltons. In certain embodiments, the polymer has an average molecular weight of about 10,000 daltons to about 30,000 daltons.

In certain embodiments, copolymers may be synthesized to achieve desired mechanical properties and release profile.

Accordingly, in certain embodiments, the poly(anhydride-ester) is a copolymer.

In certain embodiments, the copolymer comprises two or more polymers (e.g., a poly(anhydride-ester) as described herein).

As used herein, a “hydrophilic polymer” is a water-soluble polymer.

Hydrophilic polymers capable of producing physical or chemical crosslinking in general with bioactive-based polyanhydrides may be used (e.g., polyvinylpolypyrrolidone (PVPP), poly(vinyl alcohol) (PVA), polyurethane (PU) or poly(ethylene oxide)(PEO)).

Accordingly, in certain embodiments, the hydrophilic polymer comprises poly(N-vinyl-2-pyrrolidone), polyvinylpolypyrrolidone, poly(vinyl alcohol), polyurethane or poly(ethylene oxide).

In certain embodiments, the hydrophilic polymer is poly(N-vinyl-2-pyrrolidone).

In certain embodiments, the hydrophilic polymer as described herein has an average molecular weight of about 40,000 daltons to about 2,000,000 daltons. In certain embodiments, the hydrophilic polymer as described herein has an average molecular weight of about 100,000 daltons to about 2,000,000 daltons. In certain embodiments, the hydrophilic polymer as described herein has an average molecular weight of about 500,000 daltons to about 2,000,000 daltons. In certain embodiments, the hydrophilic polymer as described herein has an average molecular weight of about 750,000 daltons to about 2,000,000 daltons. In certain embodiments, the hydrophilic polymer as described herein has an average molecular weight of about 1,000,000 daltons to about 2,000,000 daltons. In certain embodiments, the hydrophilic polymer as described herein has an average molecular weight of about 1,000,000 daltons to about 1,750,000 daltons. In certain embodiments, the hydrophilic polymer as described herein has an average molecular weight of about 1,000,000 daltons to about 1,500,000 daltons. In certain embodiments, the hydrophilic polymer as described herein has an average molecular weight of about 1,300,000 daltons.

In certain embodiments, copolymers may be synthesized to achieve desired mechanical properties and release profile.

Accordingly, in certain embodiments, the hydrophilic polymer is a copolymer or blend of two or more polymers.

In certain embodiments, the two or more polymers are selected from poly(N-vinyl-2-pyrrolidone), polyvinylpolypyrrolidone, poly(vinyl alcohol), polyurethane and poly(ethylene oxide).

In certain embodiments, the ratio of the poly(anhydride-ester) to the hydrophilic polymer ranges between about 1:9 to about 1:1. In certain embodiments, the ratio of the poly(anhydride-ester) to the hydrophilic polymer ranges between about 1:9 to about 4:6. In certain embodiments, the ratio of the poly(anhydride-ester) to the hydrophilic polymer ranges between about 1:9 to about 3:7. In certain embodiments, the ratio of the poly(anhydride-ester) to the hydrophilic polymer ranges between about 1:9 to about 2:8. In certain embodiments, the ratio of the poly(anhydride-ester) to the hydrophilic polymer is about 1:9. In certain embodiments, the ratio of the poly(anhydride-ester) to the hydrophilic polymer is about 2:8. In certain embodiments, the ratio of the poly(anhydride-ester) to the hydrophilic polymer is about 3:7. In certain embodiments, the ratio of the poly(anhydride-ester) to the hydrophilic polymer is about 4:6. In certain embodiments, the ratio of the poly(anhydride-ester) to the hydrophilic polymer is about 1:1.

Additional bioactive molecules, synthetic or natural, may be incorporated in the hydrogels described herein to achieve enhanced biological or mechanical properties. For example, free bioactive molecules (e.g., curcumin) may be incorporated into the blended solution for hydrogel production, resulting in a material with dual release of bioactive molecules.

Accordingly, in certain embodiments, the hydrogel further comprises a bioactive molecule dispersed in the hydrogel.

In certain embodiments, the bioactive molecule dispersed in the hydrogel is the same as the bioactive molecule yielded by hydrolysis of the polymer backbone.

In certain embodiments, the bioactive molecule dispersed in the hydrogel is different from the bioactive molecule yielded by hydrolysis of the polymer backbone.

In certain embodiments, the bioactive molecule dispersed in the hydrogel is selected from ferulic acid, sinapic acid, coumaric acid (e.g., p-coumaric acid), salicylic acid and curcumin.

In certain embodiments the bioactive molecule dispersed in the hydrogel is curcumin.

As used herein, the term “crosslink” can refer to physical (e.g., intermolecular interactions or entanglements, such as through hydrophobic interactions) or chemical crosslinking (e.g., covalent bonding). Chemical crosslinking may be induced for these hydrogels using ultraviolet (UV) radiation, gamma radiation, an external cross-linking agent, or Fenton and photo-Fenton reactions to obtain chemical hydrogels. This chemical crosslinking may result in a more stable and non-reversible material, wherein the bioactive-based polymer is trapped within the PVP three-dimensional network.

Accordingly, in certain embodiments, the poly(anhydride-ester) is physically crosslinked with the hydrophilic polymer through hydrophobic interactions.

In certain embodiments, the poly(anhydride-ester) is chemically crosslinked with the hydrophilic polymer.

In certain embodiments, the poly(anhydride-ester) is covalently crosslinked with the hydrophilic polymer.

In certain embodiments, the poly(anhydride-ester) is crosslinked with the hydrophilic polymer using a free radical mechanism.

Certain embodiments provide a hydrogel as described herein.

As described herein, the synthesis of the hydrogels (e.g., the bioactive-based PVP/polyanhydride blended materials), may be prepared as films by solvent-cast methods and as nanofibers using electrospinning technique. As described herein, the materials may be produced at varying ratios to achieve the desired formulation.

Accordingly, certain embodiments of the invention provide a method of making a hydrogel as described herein, comprising solvent-casting (a) a poly(anhydride-ester) comprising a polymer backbone and having a group in the polymer backbone that will yield a bioactive molecule upon hydrolysis of the polymer backbone; and (b) a hydrophilic polymer; under conditions to provide a hydrogel. In certain embodiments, the solvent is DMF. In certain embodiments, the solvent is DMSO. Certain embodiments of the invention provide a hydrogel prepared by the methods described herein.

Certain embodiments provide a method of making a hydrogel as described herein, comprising electrospinning (a) a poly(anhydride-ester) comprising a polymer backbone and having a group in the polymer backbone that will yield a bioactive molecule upon hydrolysis of the polymer backbone; and (b) a hydrophilic polymer; under conditions to provide a hydrogel. Certain embodiments of the invention provide a hydrogel prepared by the methods described herein.

Certain embodiments provide a method comprising cross-linking the poly(anhydride-ester) with the hydrophilic polymer using ultraviolet radiation, gamma radiation or an external cross-linking agent.

Certain embodiments provide a method of making a hydrogel as described herein.

Certain embodiments provide a hydrogel prepared as described herein.

Certain embodiments provide a method for promoting wound healing in a mammal, comprising contacting a hydrogel as described herein with a wound of the mammal.

In certain embodiments, the wound is a burn.

Certain embodiments provide a method of therapeutically treating the skin of a mammal, comprising contacting a hydrogel as described herein with the skin of the mammal.

In certain embodiments, the mammal is a human.

In certain embodiments, the bioactive molecule yielded upon hydrolysis of the polymer backbone has an antimicrobial, anti-inflammatory, antioxidant or analgesic effect.

In certain embodiments, the poly(anhydride-ester) comprises (a) a polymer backbone and having a group in the polymer backbone that will yield a bioactive molecule upon hydrolysis of the polymer backbone; and (b) one or more linker molecules in the polymer backbone comprising one or more photoreactive double bonds.

In certain embodiments, the poly(anhydride-ester) is

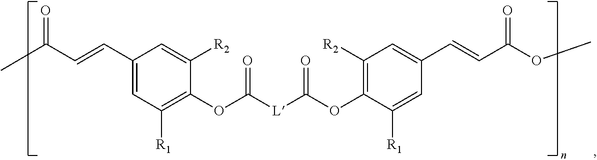

wherein
R₁ is OCH₃ and R₂ is H; R₁ is OCH₃ and R₂ is OCH₃; or R₁ is H and R₂ is H;
L′ is

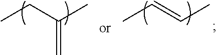

and n is 2 or more.

In certain embodiments the poly(anhydride-ester) is:

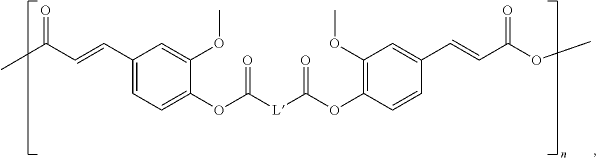

wherein L′ is

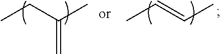

and n is 2 or more.

In certain embodiments, the poly(anhydride-ester) is

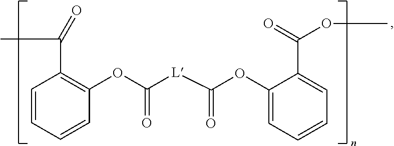

wherein
L′ is

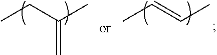

and n is 2 or more.

In certain embodiments, the hydrogel is prepared by solvent-casting a polymer described herein to generate a material and exposing the material to ultraviolet radiation.

The following definitions are used, unless otherwise described: halo is fluoro, chloro, bromo, or iodo. Alkyl, alkoxy, etc. denote both straight and branched groups; but reference to an individual radical such as “propyl” embraces only the straight chain radical, a branched chain isomer such as “isopropyl” being specifically referred to. Aryl denotes a phenyl radical or an ortho-fused bicyclic carbocyclic radical having about nine to ten ring atoms in which at least one ring is aromatic. Heteroaryl encompasses a radical attached via a ring carbon of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₆)alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloallcyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₁-C₆)allcylthio can be methylthio, ethylthio, propylthio, isopropylthio, butylthio, isobutylthio, pentylthio, or hexylthio; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

The hydrogels described herein may be used as a new wound dressing that could be applied to wounds and burns. The physical crosslinking between a hydrophilic polymer (e.g., PVP) and bioactive-based polymers is a completely new approach for wound dressing production. One of the improvements provided by this type of dressings is more comfort for the patients, as PVP hydrogels significantly decrease pain. Moreover, these blended hydrogels can release bioactive molecules upon hydrolytic degradation of the bioactive-based polyanhydride (e.g. antimicrobial, antioxidant, anti-inflammatory and analgesic agents), providing an ideal condition for tissue repair and improving the healing process.

Certain embodiments of the invention will now be illustrated by the following non-limiting Examples.

### EXAMPLE 1

Hydrogels are promising biomaterials for wound healing applications due to their unique ability to absorb large volumes of water or biological fluids without dissolving. To improve healing, bioactives are commonly chemically and physically incorporated into hydrogel networks. However, a partially biodegradable hydrogel that releases bioactive molecules upon hydrolytic degradation to allow controlled release of the drug would be advantageous. Therefore, bioactive-based polyanhydrides derived from ferulic acid, sinapic acid, p-coumaric acid, and salicylic acid were synthesized and blended with poly(N-vinyl-2-pyrrolidone) (PVP) to produce hydrogels. The bioactives offer antimicrobial, anti-inflammatory, and antioxidant properties, which when formulated into biodegradable polymers, will achieve controlled release of the bioactive upon degradation, and cross-linking with PVP allows hydrogel formation. The polymer systems were physically cross-linked via hydrophobic interactions, as found using differential scanning calorimetry and Fourier transform infrared spectroscopy. Porous structures were confirmed by scanning electron microscopy analysis of the hydrogels. In addition, the swelling ratios correlate with the polyanhydride content; a higher percentage of PVP produced hydrogels with higher swelling ratios. Additionally, the PVP/salicylic acid-based polymer hydrogels demonstrated the best swelling values as compared to the other polymers at the same ratios. This work highlights the potential of these systems for wound healing applications.

Bioactive-based polyanhydrides are unique in that the bioactive is chemically incorporated into the polymer backbone and is released upon hydrolysis in a controlled manner (Carbone A. L. Natural Bioactive-Based Polyanhydrides for Controlled Release Applications. PhD Dissertation, Rutgers, The State University of New Jersey, 2009; Prudencio A.; Schmeltzer R. C.; Uhrich K. E. Macromolecules, 2005, 38(16), 6895-6901; Carbone A. L.; Uhrich K. E. Macromol. Rapid Commun. 2009, 30(12), 1021-1026). Scheme 1 illustrates the synthesis of ferulic acid (FA), sinapic acid (SinA), p-coumaric acid (p-CA), and salicylic acid (SAA) polymers with an adipic linker via solution polymerization. The formulated biodegradable polymers were physically cross-linked with PVP to allow hydrogel formation. Upon degradation, the bioactives released offer antimicrobial, anti-inflammatory, and antioxidant properties.

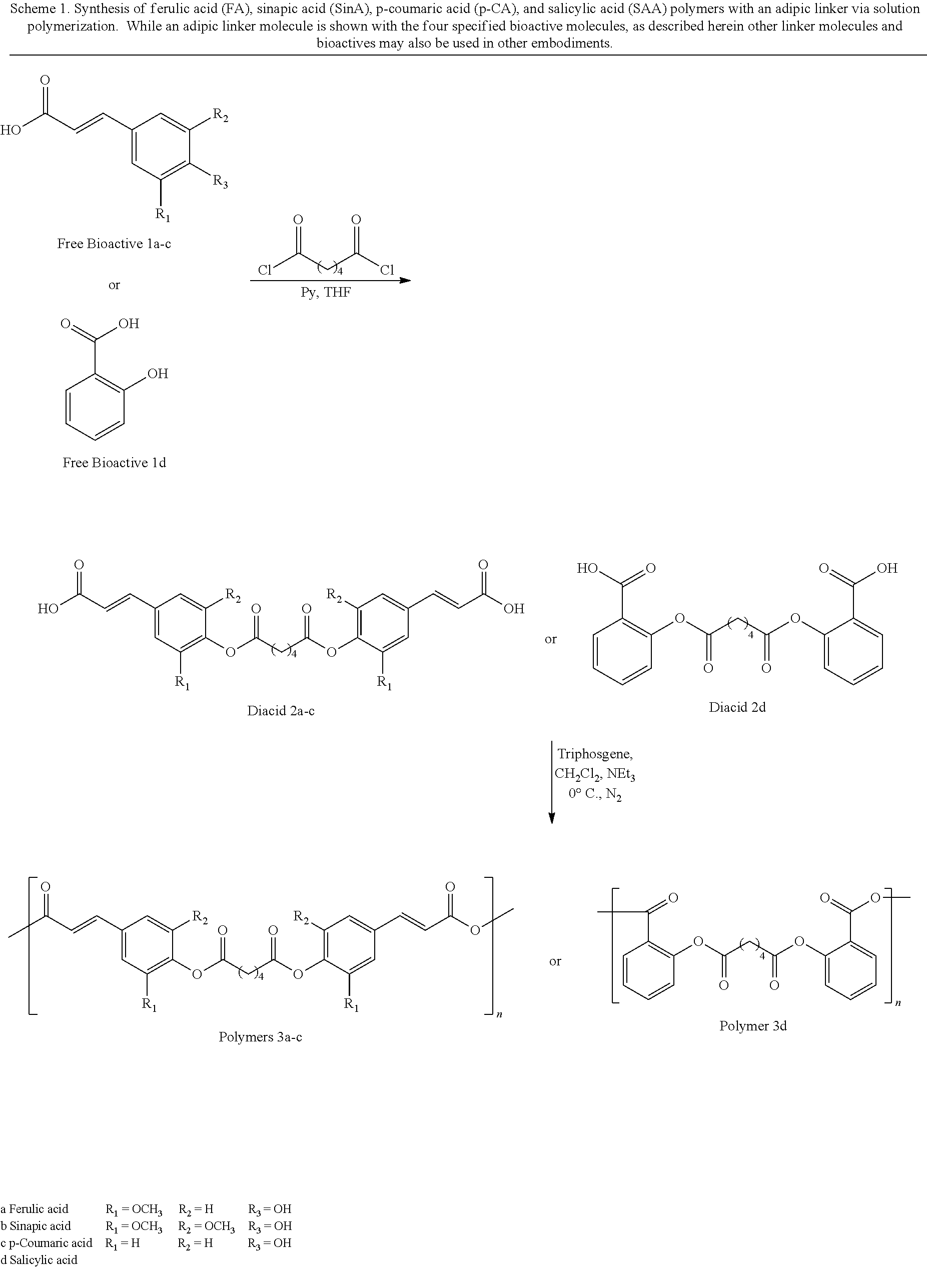

The hydrogels were generated as shown in FIG. 1. Glass transition temperatures (T_{g}) of the films were obtained by differential scanning calorimetry. A single T_{g} value confirms all produced hydrogels are comprised of miscible blended materials (Table 1).

TABLE 1
Glass transition temperatures (T_{g}) for the blended membranes
of bioactive-based PVP/polyanhydride films.

| Sample | Ratio | T_{g} (° C.) |
|---|---|---|
| PVP | 1:0 | 182 |
| PVP/FA | 0:1 | 58 |
| PVP/FA | 9:1 | 130 |
| PVP/FA | 8:2 | 115 |
| PVP/FA | 7:3 | 90 |
| PVP/SinA | 0:1 | 120 |
| PVP/SinA | 9:1 | 140 |
| PVP/SinA | 8:2 | 125 |
| PVP/SinA | 7:3 | 115 |
| PVP/p-CA | 0:1 | 60 |
| PVP/p-CA | 9:1 | 155 |
| PVP/p-CA | 8:2 | 138 |
| PVP/p-CA | 7:3 | 100 |
| PVP/SAA | 0:1 | 46 |
| PVP/SAA | 7:3 | 68 |

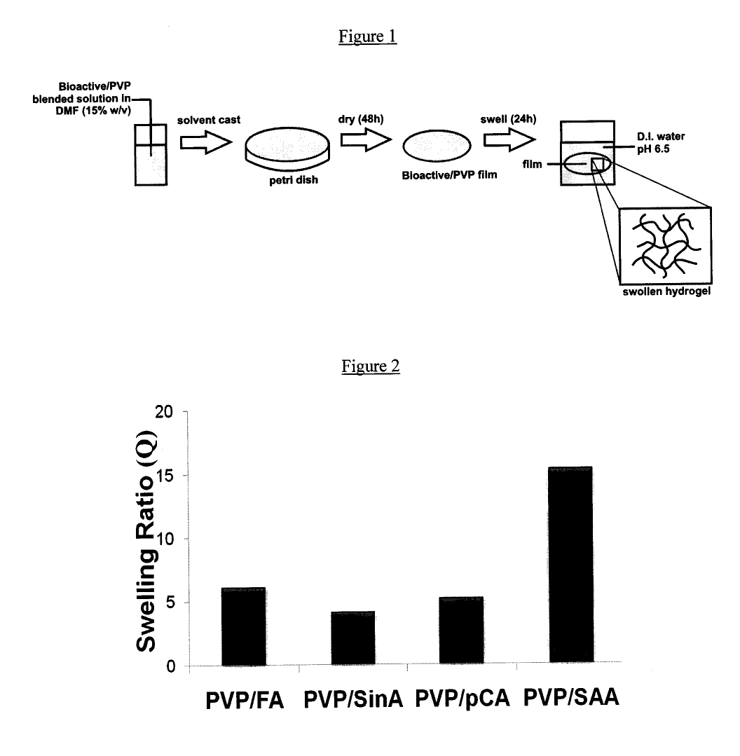

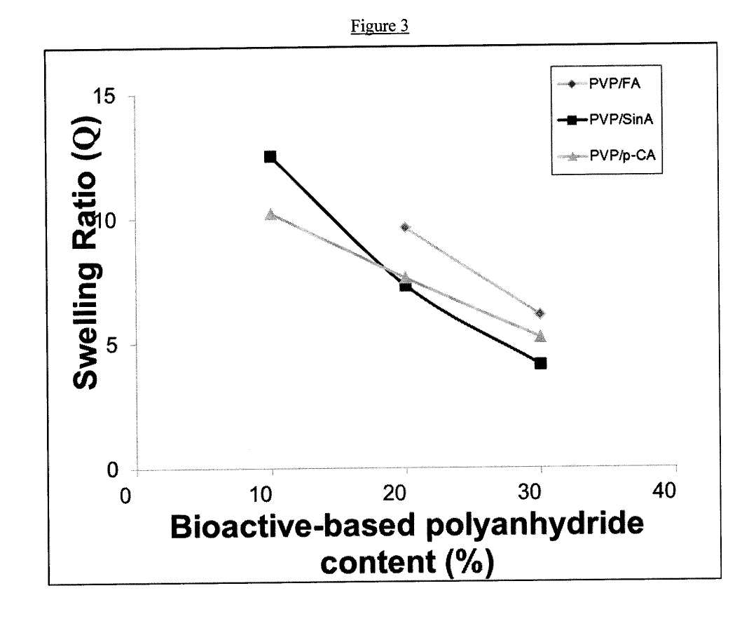

The swelling ratio (Q) was calculated after the films were placed in deionized water for 24 h using the following formula: Q=(Wₛ−W_{d})/W_{d}, where Wₛ and W_{d} are the weight of swollen and dried gels respectively. Swelling ratios correlate with the bioactive content of the hydrogels, where better swelling ratios were found with the hydrogels of higher PVP content (FIG. 2). Best water uptake capability was seen with PVP/SAA hydrogels compared to the other bioactive-based hydrogels of the same ratio (FIG. 3).

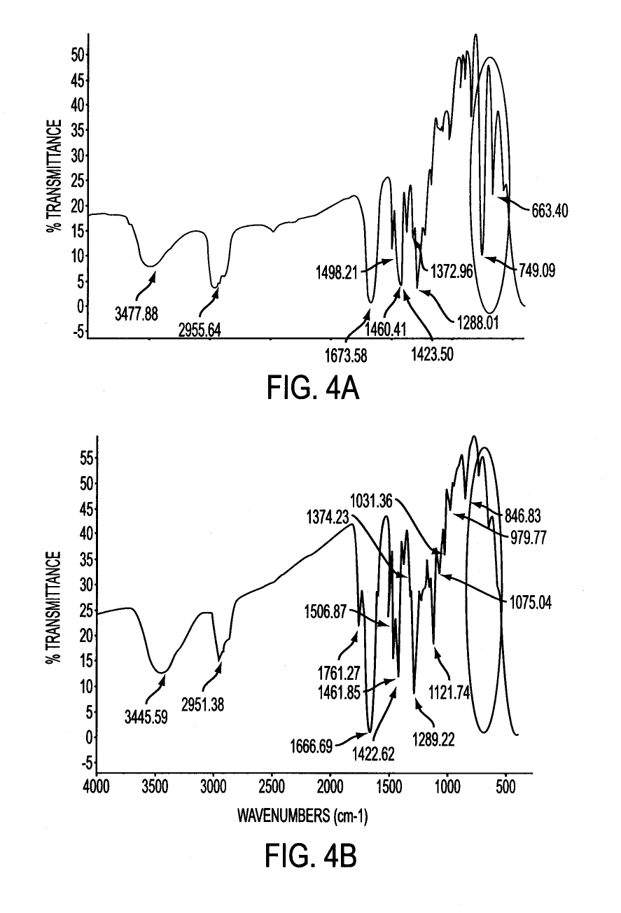

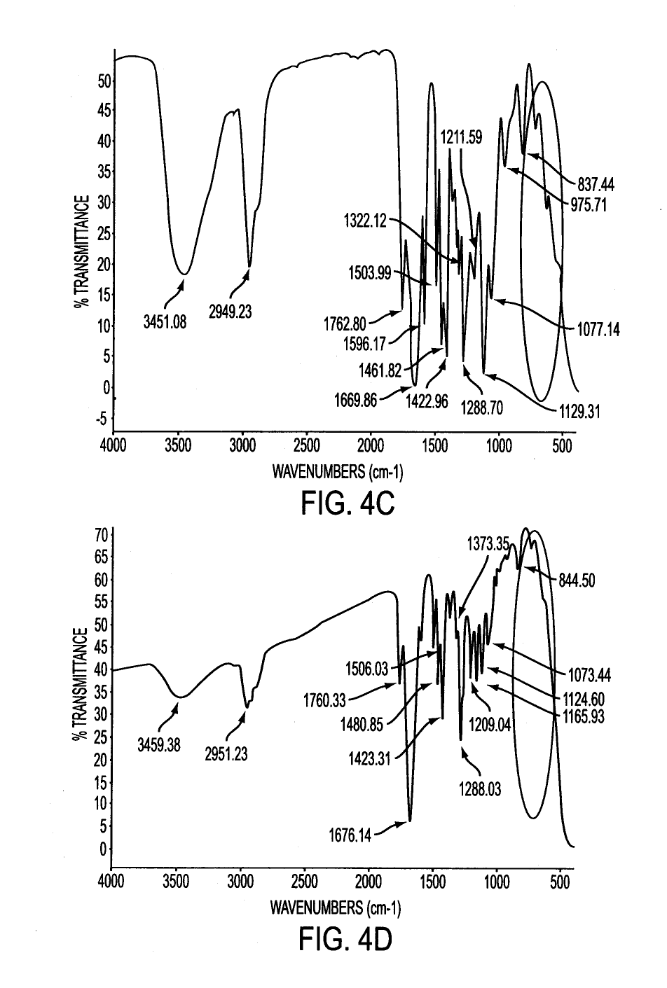

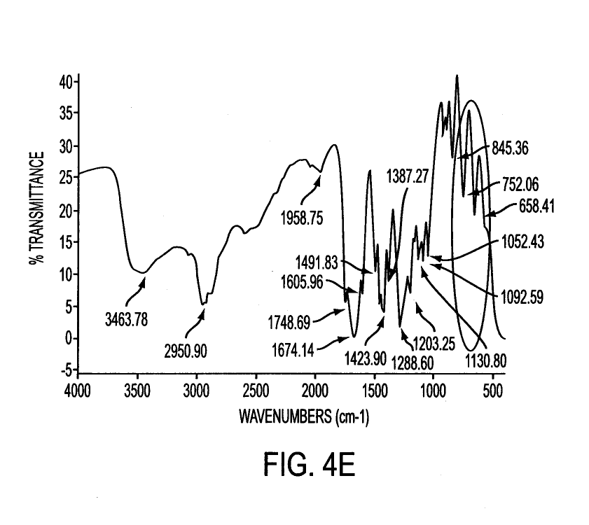

Fourier transform infrared (FTIR) spectroscopy was used to examine the chemical interactions between the blended polymers. FTIR spectra (FIG. 4) reveal hydrophobic interactions are responsible for the miscibility and physical cross-linking. Increased hydrophobic interactions create ordered domains causing a reduction in the stretching capability of the hydrogel. A strong decrease in intensity of the hydrophobic region (CH₂ rock, circled in the spectra shown in FIG. 4) for all blended materials was observed, but less significant in PVP/SAA due to its higher hydrophilicity.

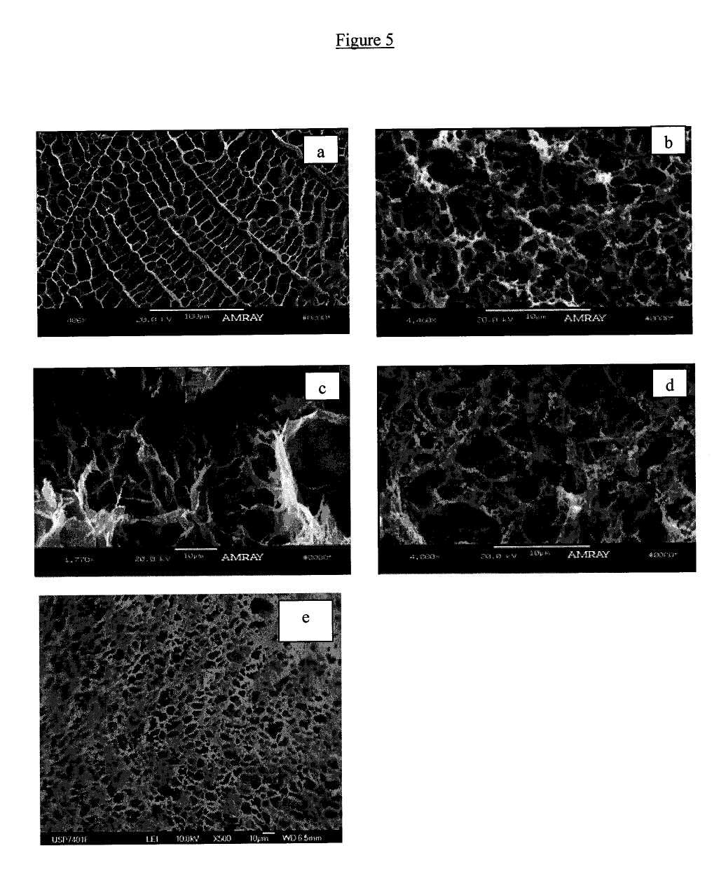

Scanning electron microscopy (SEM) was used to monitor the morphology of the hydrogels. The SEM images (FIG. 5) reveal a porous structure for all PVP/bioactive blended hydrogels. The hydrogel's highly porous structure is useful for wound healing purposes. PVP/FA and PVP/SinA hydrogels display a more organized porous structure.

Physically cross-linked hydrogels of various ratios were successfully produced as bioactive-based polyanhydride/PVP blends with good swelling ratios, where hydrophobic interactions are responsible for the hydrogel formation. The swelling degrees correspond to the hydrophobic and hydrophilic nature of the polyanhydride used. PVP/SAA (7:3) displayed the best overall swelling degree. All bioactive-based polymers produced miscible blended hydrogels with PVP. The porous structure is beneficial for wound dressing applications to enhance the healing process by promoting gas exchange and drug delivery.

### EXAMPLE 2

Hydrogels are tridimensional hydrophilic polymeric networks that can absorb large volumes of water and biological fluids, making them promising materials for biomedical applications (e.g., wound dressings and skin care therapeutics). A series of bioactive polymers derived from ferulic acid, sinapic acid, p-coumaric acid, and salicylic acid were synthesized and combined with poly(N-vinyl-2-pyrrolidone) (PVP) to produce novel blended hydrogels. The goal of these systems is to utilize the antimicrobial, antiinflammatory, and antioxidant properties of the bioactive, formulate them into biodegradable polymers to achieve controlled release of the bioactive, and obtain the unique properties hydrogels offer by utilizing PVP. Differential scanning calorimetry, fourier transform infrared spectroscopy, and scanning electron microscopy were used to characterize the resulting hydrogel blends. In addition, the swelling ratios of the blends were also evaluated and found to correlate with the bioactive polymer content. PVP/salicylic acid-based polymer hydrogels demonstrated the best swelling degree values.

Hydrogels are a well known promising class of biomaterials with a large field of applications including contact lenses, drug delivery systems, scaffolds for tissue engineering, and wound dressings (Peppas et al., Annu. Rev. Biomed. Eng. 2000, 2, 9-29). They are comprised of a hydrophilic polymer network, which can absorb large volumes of water and biological fluids without dissolving. In addition, some hydrogels can respond to changes in pH, an electrical field, and temperature or possess antimicrobial, anti-inflammatory, and antioxidant properties (Peppas et al. Annu. Rev. Biomed. Eng. 2000, 2, 9-29; Abd El-Mohdy H. L. et al., J. Polym. Res. 2009, 16, 1-10; Trombino S. et al., Carbohydrate Polymers 2009, 75, 184-188; Su J. et al., Biomaterial 2010, 31(2), 308-314). Accordingly, in certain embodiments the hydrogels disclosed herein are useful for such applications.

Poly(N-vinyl-2-pyrrolidone) (PVP) is a biocompatible polymer that has been used to produce hydrogels (e.g., by chemically crosslinking; does not form physically cross-linked hydrogels alone) and materials for biomedical applications (Lopergolo L. C., et al., Polymer 2003, 44, 6217-6222; Barros J. A. G. et al., Polymer 2006, 47, 8414-8419). PVP is an amphiphilic polymer, which can interact with hydrophilic polymers such as polyvinyl alcohol and with hydrophobic polymers such as poly(D,L-lactide). Hydrogels for wound dressings comprised of PVP and poly(ethylene glycol), agar, and water are commercially available (HDR® and AQUAGEL®). Common methods to prepare PVP hydrogels include water radiolysis, gamma radiation, electron beam radiation and ultra violet radiation. However, these high energy methods limit the type of drugs that can be incorporated to enhance wound healing. Specifically, if loaded before irradiation, the bioactivity of the drug could be compromised, and if loaded after irradiation, the type of drug and loading capabilities are limited.

However, as described herein, PVP hydrogels incorporated with bioactive polymers could be used as wound dressings to improve the healing process releasing active molecules, while the soft hydrogel structure provided by PVP can keep an ideal humidity, which results in lower adhesion and reduced damage to new tissue formation. Accordingly, the production of hydrogels made of blends composed of bioactive based polyanhydrides and poly(N-vinyl-2-pyrrolidone) (PVP) are described herein. Bioactive-based polyanhydrides such as ferulic acid (FA), sinapic acid (SinA), p-coumaric acid (p-CA) and salicylic acid (SAA) adipic polymers may be used to produce miscible blends with PVP, resulting in hydrogels. These hydrogels are unique in that they provide a combined advantage offered by PVP hydrogels including softness, absorbency, transparency, biocompatibility, and mechanical properties with the bioactive-based polyanhydrides, which release bioactive molecules and a biocompatible linker by hydrolytic degradation. Moreover, the hydrogels are easy to produce by blending both polymers, which leads to a physical crosslinking and in turn allows the incorporated bioactive to remain undamaged. This physical cross-linking by hydrophobic interactions between PVP and the bioactive-based polyanhydrides also results in enhanced stability compared to other physical hydrogels previously described. The water absorbed by these hydrogels would be responsible for the hydrolytic degradation, where the degradation products include the bioactive molecules that possess antimicrobial, antioxidant, and anti-inflammatory activity as well as a biocompatible linker.

A series of bioactive polymers derived from ferulic acid, sinapic acid, p-coumaric acid, and salicylic acid have been synthesized and explored as bioactive delivery systems. These bioactive polymers are unique in that the drugs are chemically incorporated into the polymer backbone. Upon hydrolysis, the drugs, which have antimicrobial, anti-inflammatory, and antioxidant properties, are released in a controlled manner.

This Example describes the production of novel blended hydrogels made of PVP and the aforementioned bioactive polymers, and investigates their properties for applications as wound dressings and skin care therapeutics.

PVP Blended Hydrogels. Ferulic acid (FA), sinapic acid (SinA), p-coumaric acid (p-CA), and salicylic acid (SAA) polymers, as shown in the schematic below, were synthesized according to previously described methods (Carbone A. L. Natural Bioactive-Based Polyanhydrides for Controlled Release Applications. PhD.Dissertation, Rutgers, The State University of New Jersey, New Brunswick, N.J., October, 2009; Prudencio A.; Schmeltzer R. C.; Uhrich K. E. Macromolecules, 2005, 38(16), 6895-6901). Solutions of PVP and the above mentioned polymers (15% w/v) were prepared in anhydrous DMF in varying ratios and allowed to stir for 24 h. The resulting films were cast onto glass plates and dried at room temperature. Other solvents may also be used, such as DMSO. The use of alternative solvents may be used to alter the properties of the resulting hydrogel. The structures of the bioactive PVP-based hydrogels are shown below.

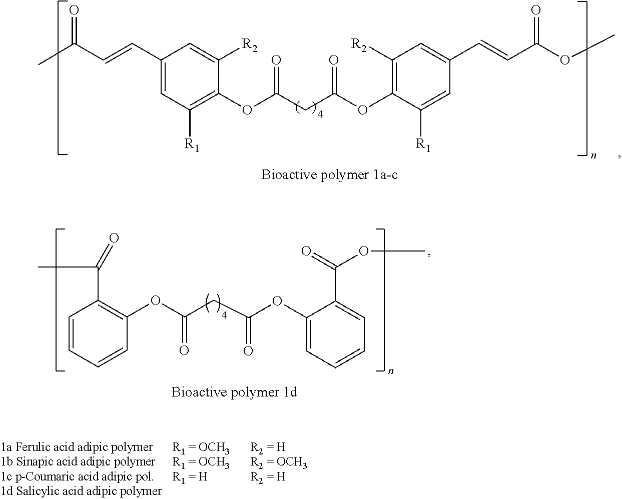

Hydrogel Characterization. Differential scanning calorimetry (DSC, Thermal Advantage Q200 system) was performed to characterize the hydrogel membranes and evaluate the miscibility from the glass transition temperatures (T_{g}). The samples were analyzed with heating under nitrogen gas from −10° C. to 200° C. at a rate of 10° C./min followed by cooling at 200° C. to −10° C. at a rate of 10° C./min using a minimum of two cycles. Chemical changes were examined by fourier transform infrared (FT-IR, Thermo Nicolet/Avatar 360) spectra of samples solvent-casted onto NaCl plates. The swelling ratio (Q) was measured after the hydrogels were placed in deionized water for 24 h (pH 6.5, 25° C.). The water uptake was measured according to Equation 1, where wₛ and w_{d} are the weight of swollen and dried hydrogels respectively.

$$Q = \frac{{w}_{s} - {w}_{d}}{{w}_{d}} \qquad Equation 1$$

The morphology of the hydrogels was monitored using scanning electron microscopy (SEM, Amray 1830 I). Membranes were allowed to swell and then lyophilized to keep their structure.

Bioactive PVP-based hydrogels were successfully produced by solvent casting. Additional cross-linking procedures (such as UV or gamma radiation) were not necessary as all bioactive polymers used formed a tridimensional network with PVP.

DSC is a useful technique to determine if a desired blend is miscible or not. If no more than one glass transition temperature is observed for a blended material, this is a strong indication of the miscibility for blended compounds. As shown in Tablel in Example 1, all bioactive polymers produced miscible blended hydrogels with PVP as evidenced by the observation of a single glass transition temperature for each material.

The main peaks indicating the structure of the bioactive polymers and PVP alone were observed in the FT-IR spectra of the blended hydrogels. Changes in the CH and CH₂ stretching in the PVP region were observed in all samples. The PVP peak at 749 cm⁻¹ (amide V or CH₂ rock peak) was no longer visible, and a shift and decrease in the intensity of the peak at 660 cm ⁻¹ (amide IV) was observed. It is well known that PVP can form strong hydrogen bonds that could create cross-linking within the polymer matrix. The hydrophobic bioactive polymers do not hydrogen bond, which may indicate the involvement of hydrophobic interactions between the bioactive polymers and the amphiphilic PVP.

Due to the hydrophobic structure of the bioactive polymers, the swelling ratio for the hydrogels (Table 2) was not comparable to the PVP hydrogel alone (with the exception of PVP/SAA). Nonetheless, these hydrogels are very significant and promising since the chemical properties of the bioactive polymers can be modified. This can be achieved by changing the linker molecule of the bioactive polymer to create more hydrophilic or hydrophobic systems (Prudencio A. Et al., Macromolecules, 2005, 38(16), 6895-6901; Carbone A. L., et al., Macromol. Rapid Commun. 2009, 30(12), 1021-1026). Although the swelling ratios were not as high as PVP alone, the bioactive PVP-based hydrogels are comprised of a more robust material i.e., can be stretched without breaking when physically manipulated. This type of structure is beneficial for the aforementioned applications.

PVP/SAA-based polymer hydrogels demonstrated swelling degrees (Table 2) that are comparable to PVP hydrogels (Lopergolo L. C. et al., Polymer 2003, 44, 6217-6222; Barros J. A. G., et al., Polymer 2006, 47, 8414-8419). Due to their increased hydrophilicity (compared to the other bioactive polymers), the PVP/SAA hydrogels exhibited faster swelling than the other PVP/bioactive polymer hydrogels.

TABLE 2
Swelling Ratio (Q) for PVP/Bioactive Hydrogels after 24 h

| Sample | Ratio | Q |
|---|---|---|
| PVP/FA | 9:1 | — |
| PVP/FA | 8:2 | 9.6 |
| PVP/FA | 7:3 | 6.1 |
| PVP/SinA | 9:1 | 12.5 |
| PVP/SinA | 8:2 | 7.3 |
| PVP/SinA | 7:3 | 4.1 |
| PVP/p-CA | 9:1 | 10.2 |
| PVP/p-CA | 8:2 | 7.6 |
| PVP/p-CA | 7:3 | 5.2 |
| PVP/SAA | 7:3 | 15.3 |

SEM images (FIG. 5) reveal the very porous hydrogel structures with pore sizes on the micro scale. A more organized porous structure was observed for PVP/FA (FIG. 5a) and PVP-SinA (FIG. 5b) hydrogels. Porous material was also obtained from PVP/p-CA (FIGS. 5c) and PVP/SAA hydrogels (FIG. 5d). The porous structure of these hydrogels would be beneficial for wound dressing applications to ultimately enhance the healing process by promoting gas exchange and drug delivery.

The soft hydrogel structure provided by PVP used in conjunction with a biodegradable, bioactive based polymer can release the bioactives over time in a controlled manner. This can be used to enhance wound dressings and skin care therapeutics.

### EXAMPLE 3

Bioactive-based PAEs are novel degradable materials researched over the past decade. Compared to other biodegradable polymers, they are unique in that bioactive molecules are chemically incorporated into the polymeric backbone, rather than physically admixed. Upon hydrolytic degradation, the bioactive and biocompatible linker molecules are released in a controlled, sustained manner. These can be manipulated into discs, fibers, and microspheres, contributing to their widespread study as drug delivery systems.

A PAE-based hydrogel system can be used to overcome limitations of current hydrogel systems by controlling drug release and allowing high drug loading. Hydroxycinnamates (HCs) are a class of molecules with photoreactive double bonds that exhibit antioxidant and antimicrobial properties. Chemical structures of selected HCs for chemical incorporation into a polymer backbone are shown below. Incorporating bioactive molecules with photoreactive double bonds into a polymer backbone should enable chemical crosslinking.

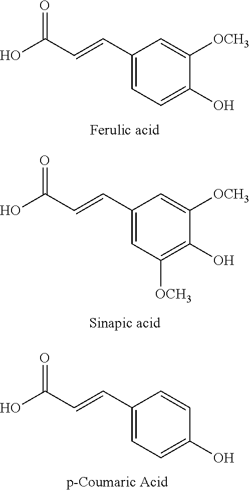 [SMILES: COc1cc(/C=C/C(=O)O)ccc1O ; COc1cc(/C=C/C(=O)O)cc(OC)c1O ; O=C(O)/C=C/c1ccc(O)cc1]

The ability to crosslink can enable the formulation of bioactive-based PAEs into hydrogels for a variety of controlled, sustained drug delivery applications thus overcoming current limitations of tunable drug release.

### Synthesis and Characterization of HC-Based PAEs

A series of HC-based polymers derived from ferulic acid, sinapic acid, and p-coumaric acid were synthesized.

Synthesis of HC-based PAEs via Solution Polymerization. HC-based PAEs were synthesized using methods shown in Scheme 2 (Schmeltzer, et al., J Biomater Sci Polym Ed 2008, 19, (10), 1295-306). Briefly, the HC (1) was dissolved in tetrahydrofuran (THF) and pyridine was then added. Adipoyl chloride was added dropwise and stirred for 24 hours. This was quenched into acidic water and the product (2) isolated via vacuum filtration. Diacid (2) was dissolved in anhydrous dichloromethane (DCM) under argon. Triethylamine (TEA) was added and the reaction cooled in an ice bath. Triphosgene, dissolved in anhydrous DCM, was added dropwise. The reaction was left to stir in an ice bath for 3 hours. This was precipitated with diethyl ether, dissolved in DCM, and washed with acidic water. After concentrating, the polymer was isolated by again precipitating with diethyl ether to remove low molecular weight oligomers.

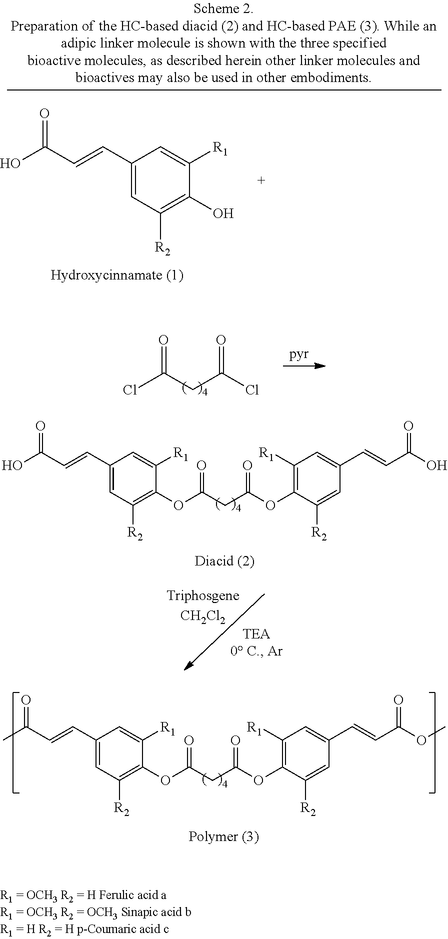

Characterization of HC-based Diacids and PAEs. PAEs and diacids were characterized using nuclear magnetic resonance (NMR) (¹H and ¹³C) and Fourier transform infrared (FTIR) spectroscopies. The melting point (Tₘ) and glass transition temperature (T_{g}) were obtained via differential scanning calorimetry (DSC), decomposition temperature using thermogravimetric analysis, and molecular weight by gel permeation chromatography methods.

### Adjusting Drug Release Profile with Admixtures

A primary goal of drug delivery systems is maintaining drug concentrations at therapeutic levels for sustained periods of time. For this purpose, hydrolytically degradable polymer systems have been used to encapsulate bioactive molecules which are released upon polymer degradation. Physical admixtures within degradable polymers generally result in a burst release of the drug from bulk eroding polymers such as poly(lactic-co-glycolic acid). Polyanhydrides, on the other hand, are hydrolytically degradable polymers that exhibit near zero-order release of encapsulated molecules in vitro due to their surface eroding properties. With SA-based PAEs, however, this linear profile is observed after a lag period of minimal SA release within the first few days. Overcoming this lag period is important for applications where both immediate and sustained release is desired. SA-based PAE admixtures that combine the burst release properties of admixtures with the high loading and sustained release properties of SA-based PAEs can be prepared as shown below.

Polymer Synthesis and Disc Preparation. SA-based PAEs (6) were synthesized with previously described methods according to synthetic Scheme 3 using adipoyl chloride as the acyl chloride (Schmeltzer, et al., Biomacromolecules 2005, 6, (1), 359-67; Erdmann, et al., Biomaterials 2000, 21, (19), 1941-6; Schmeltzer, et al., Polymer Bulletin 2003, 49, 441-448). SA (4), SA-based diacid (5), and 4:5 (1:1) mixtures were separately incorporated into the polymer at 0, 1, 5, and 10% (w/w) (Table 3) and ground with mortar and pestle. Polymer discs were prepared by pressing the ground polymer mixtures into discs.

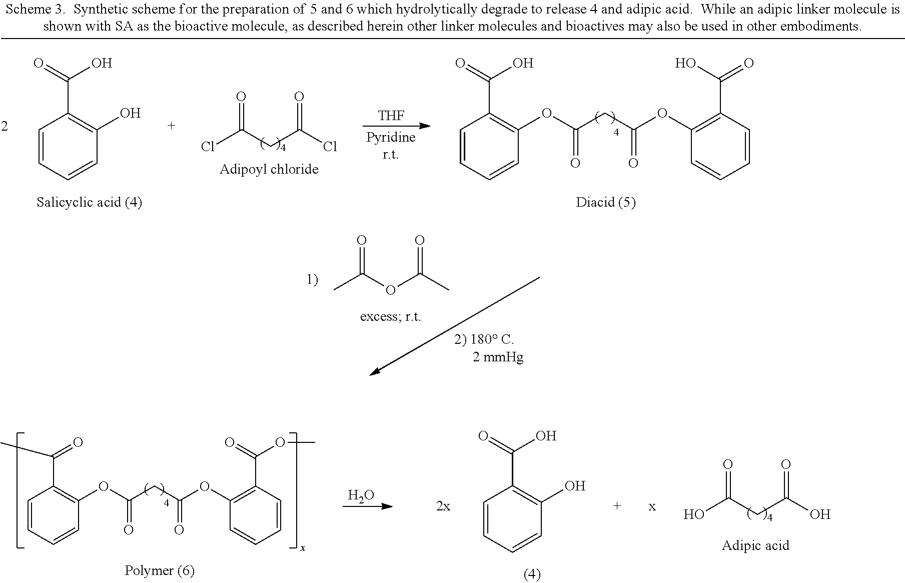

TABLE 3
Sample Compositions

| Admixture | Admixture wt % | Polymer (6) |
|---|---|---|
| Polymer Alone | 0 | a |
| SA | 1 | b |
| | 5 | c |
| | 10 | d |
| SA-based Diacid | 1 | e |
| | 5 | f |
| | 10 | g |
| SA:Diacid | 1 | h |
| (1:1) | 5 | i |
| | 10 | j |

In Vitro Hydrolytic Degradation. Sample discs were placed in 20 mL glass vials containing 10 mL phosphate buffered saline (PBS) under simulated physiological conditions (i.e., pH 7.4 and 37° C.) with continuous shaking at 60 rpm for 30 days. Media was collected and replaced with 10 mL of fresh PBS at set time points. The spent media was analyzed by ultraviolet (UV) spectrophotometry to monitor SA release. Absorbance measurements were obtained at λ=303 nm and calculated against a predetermined calibration curve. All experiments were performed in triplicate.

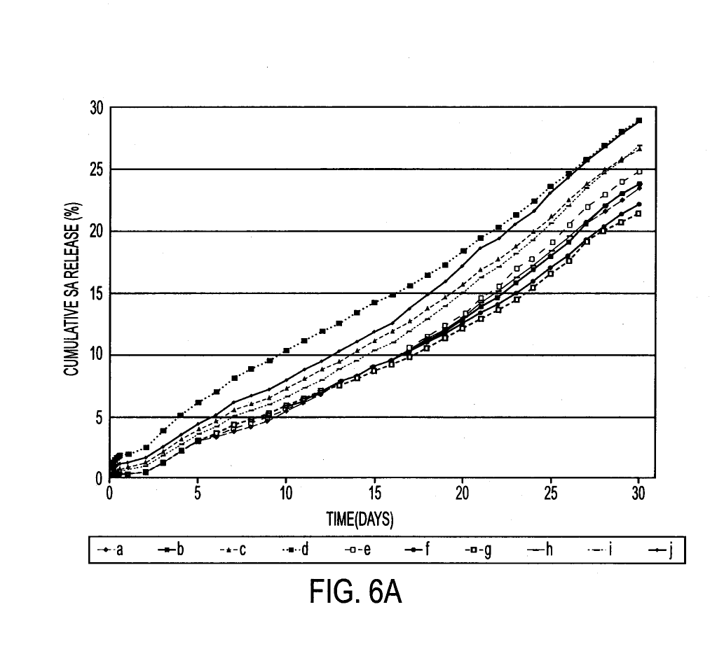

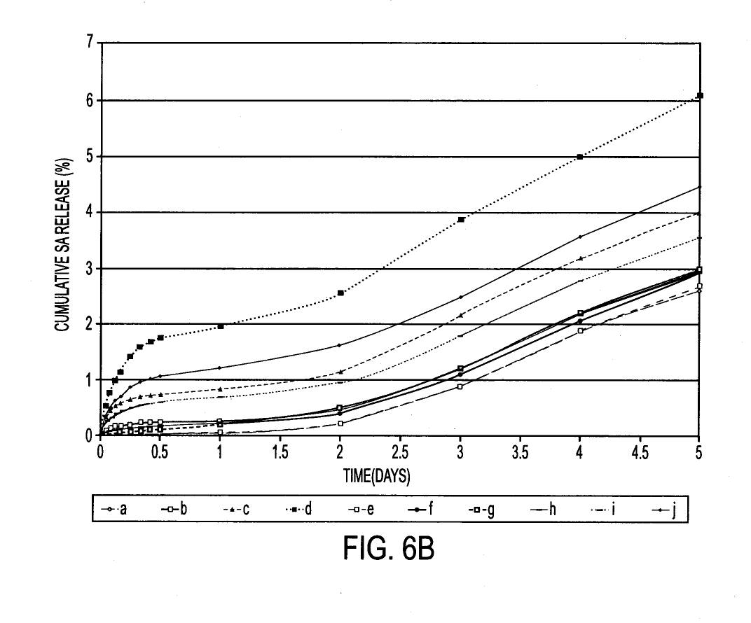

A short lag time of ˜2 days was observed for polymer alone (FIG. 6). This lag time was overcome by the admixtures, correlating with expected release based on admixture percentages. Admixtures of 4 exhibited the largest burst release profiles for their respective weight percentages, followed by the 4:5 and 5. The initial burst release did not have a significant effect on the subsequent drug release rate.

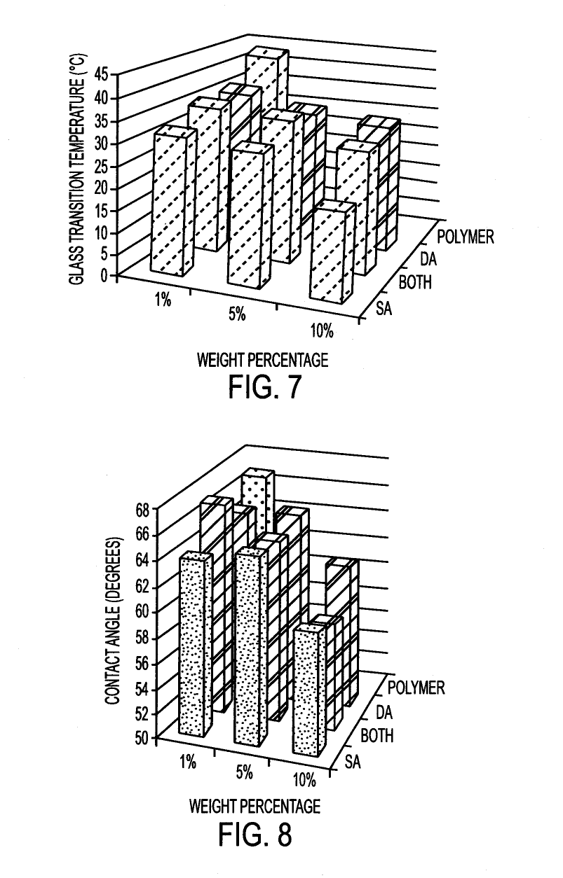

Influence of Admixtures on Polymer Properties. Physical incorporation of molecules into a polymer matrix may alter the inherent properties of the matrix. Therefore, DSC analyses were performed to determine if changes to the polymer's T_{g} were observed. The data in FIG. 7 depicts an observed decrease in T_{g} as admixture weight percentages increased, consistent with previously reported results on diffusion-controlled drug release from polymers.

Changes to the hydrophobic nature of the polymer surface were identified via static contact angle measurements with deionized water using compression-molded discs. As the admixture weight percentages increased, the contact angles decreased (FIG. 8) indicating decreased hydrophobicity of the polymer surface.

By varying the amounts and types of molecules admixed, drug delivery systems can be prepared to release SA in well-defined release profiles. The size of the burst release can be controlled by adjusting the type and ratios of admixed molecules.

### Formulation of HC-Based PAEs into Hydrogels

Every year, thousands of patients require hospitalization for severe bum injuries resulting in high morbidity and mortality rates. Poly(vinyl pyrrolidone) (PVP) is an amphiphilic, biocompatible polymer commonly used to produce hydrogels as wound dressing, but lacks inherent bioactivity. PVP hydrogels blended with HC-based PAEs would be advantageous as wound dressings with PAEs releasing bioactives to enhance healing, by forming a three-dimensional network without external crosslinking processes.

PVP Blended Hydrogels. Structures of PVP and HC-based PAEs used for the preparation of PVP:PAE hydrogel blends are shown below. The HC-based PAEs were synthesized according to the synthetic scheme for preparation of the HC-based diacid (2) and HC-based PAE (3) shown above. Solutions of PVP and aforementioned PAEs were prepared at varying ratios in anhydrous dimethylformamide and stirred for 24 h (15% w/v total polymer). The resulting solutions were cast onto Teflon plates and dried at room temperature to form films.

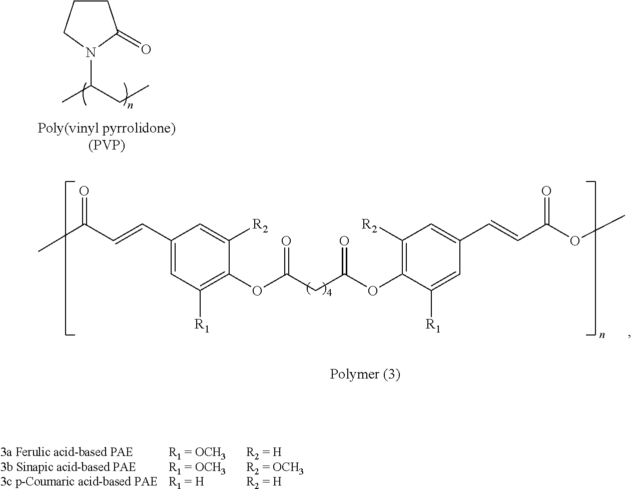

Hydrogel Characterization. All PAEs produced miscible film blends with PVP indicated by a single T_{g} observed for each material (Table 4).

TABLE 4
T_{g} Values for HC-based PVP:PAE Film Blends

| Sample | Ratio | T_{g} (° C.) |
|---|---|---|
| PVP | 1:0 | 182 |
| PVP: 3a | 0:1 | 58 |
| PVP: 3a | 9:1 | 130 |
| PVP: 3a | 8:2 | 115 |
| PVP: 3a | 7:3 | 90 |
| PVP: 3b | 0:1 | 120 |
| PVP: 3b | 9:1 | 140 |
| PVP: 3b | 8:2 | 125 |
| PVP: 3b | 7:3 | 115 |
| PVP: 3c | 0:1 | 60 |
| PVP: 3c | 9:1 | 155 |
| PVP: 3c | 8:2 | 138 |
| PVP: 3c | 7:3 | 100 |

The hydrogels were placed in deionized water for 24 hours (pH 7.4, 25° C.) and swelling ratios (Q) calculated according to Equation 1, where wₛ and w_{d} are the weight of swollen and dried hydrogels respectively.

$$Q = \frac{{w}_{s} - {w}_{d}}{{w}_{d}} \qquad Equation 1$$

The swelling ratios for the hydrogel blends (Table 5) were not as high compared to the PVP hydrogel alone (Q=15-32 depending on extent of chemical crosslinking) likely due to the hydrophobicity of the PAEs.

TABLE 5
Swelling Ratio (Q) for PVP:PAE Hydrogels after 24 h

| Sample | Ratio | Q |
|---|---|---|
| PVP: 3a | 9:1 | — |
| PVP: 3a | 8:2 | 9.6 |
| PVP: 3a | 7:3 | 6.1 |
| PVP: 3b | 9:1 | 12.5 |
| PVP: 3b | 8:2 | 7.3 |
| PVP: 3b | 7:3 | 4.1 |
| PVP: 3c | 9:1 | 10.2 |
| PVP: 3c | 8:2 | 7.6 |
| PVP: 3c | 7:3 | 5.2 |

The morphology for all 7:3 hydrogels was determined using scanning electron microscopy (SEM). Films were swelled in deionized water for 24 hours then lyophilized. SEM images revealed micrometer-sized porous hydrogel structures for all samples. These structures are beneficial for wound dressing applications by promoting gas exchange and drug diffusion to enhance healing.

A gel-like material was formed when the ferulic acid-based PVP:PAE (7:3) hydrogel film was swelled but qualitatively was weak and tore easily when stretched. Therefore, a more robust material would be useful for extended use as a wound dressing. The soft hydrogel structure provided by PVP used in conjunction with biodegradable, HC-based PAEs were prepared.

### EXAMPLE 4

Degradable HC-based PAEs with tunable physicochemical properties and controlled, sustained drug release for use as drug delivery systems for a wide variety of applications may be made as provided herein. As described below, the PVP:PAE physical hydrogels described above may be modified and optimized to enhance the hydrogel drug release profiles and mechanical properties.

### Modifying the Ccemistry of the PAE will Significantly Alter the Degradation Profile of the Blended Hydrogel, Thus Changing Drug Release

One of the parameters to control current drug release involves the hydrophobicity of the system. A different linker will allow the hydrophilicity of the PVP:PAE-based hydrogel to be altered, thus modifying drug release. The effect of linker structure on bioactive-based PAEs has been studied extensively and has been implemented herein (Prudencio, et al., Macromolecules 2005, 38, (16), 6895-6901; Carbone, et al., Macromol Rapid Commun 2009, 30, (12), 1021). For example, a more hydrophilic linker, diglycoyl choride, may be utilized instead of a hydrophobic adipoyl chloride linker, to produce blended hydrogels with a faster release of the bioactive molecules. A linker containing double bonds may also be used.

When using the largest amount of PAE (7:3 PVP:PAE ratio), 3a, ferulic acid-based PAE, had the highest swelling ratio (Q=6.1) (described above). Accordingly, Ferulic acid is used as an example bioactive below; however, other bioactives may be used.

Preparation of Hydrogel Blends.

Changing Chemical Composition of PAE via linker molecule: Branched aliphatic, linear aliphatic, and oxygen-containing linker molecules can be used to control the release rate of ferulic acid from the hydrogel blends. Diethylmalonyl, adipoyl, and diglycolyl chlorides will be used as representative molecules for these respective groups.

Solvent Casting Blends: Synthesis of 8 will be performed to determine linker effect on drug release rate (Scheme 4). Once synthesized, the PAEs will be dissolved with PVP in a mutual solvent, cast onto Teflon plates, and dried under vacuum.

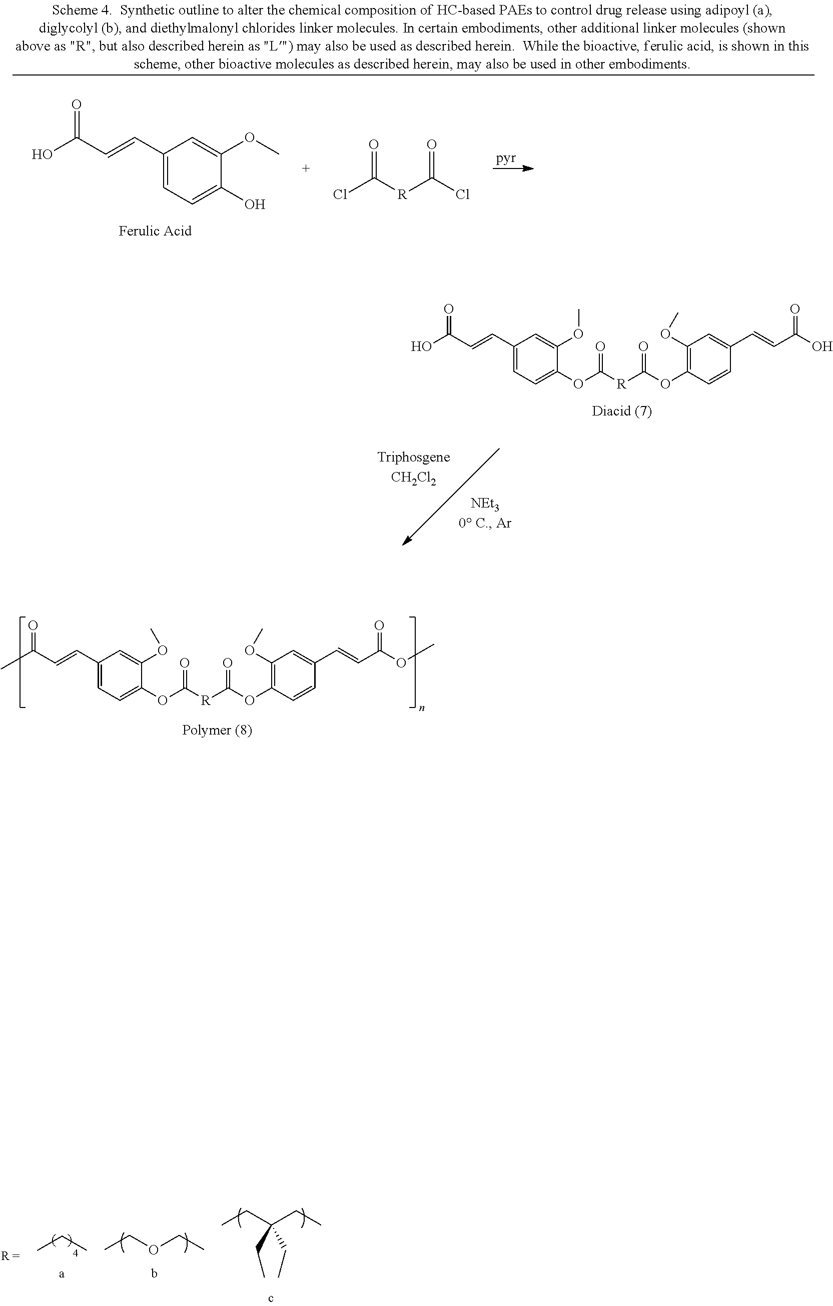

Characterization of Hydrogel Blends.

DSC: Because phase separation can alter the drug release profile, the miscibility of each blend will be determined using its T_{g} where one T_{g} indicates a miscible blend.

Swelling Ratio: Water uptake will be determined using Equation 1, described above, to determine the maximum amount of water the hydrogel can absorb. The diethylmalonic linker may be too hydrophobic to swell at 7:3 PVP:PAE and PVP ratio could then be increased.

IR: When the carbonyl group of PVP (peak between 1650 and 1680 cm⁻¹) form intermolecular bonding, there is a negative shift exhibited in the IR spectrum. The intermolecular interactions between PVP and PAE will be determined based upon these shifts.

SEM: The morphology and porosity of the hydrogels will be determined by SEM using procedures outlined above.

In Vitro Drug Release from Hydrogel Blends. It is important to not only prepare materials with enhanced properties, but to ensure that the bioactive is released at a desired rate. This can be performed via in vitro studies in PBS under simulated physiological conditions outlined above.

UV: A wavelength to analyze drug release will be chosen based upon one that does not overlap with the absorbance profiles of other degradation products (e.g. PVP, adipic acid, and PAE oligomers). Once chosen, the amount of HC released at a specific time point will be quantified against a predetermined calibration curve.

High Performance Liquid Chromatography (HPLC): If absorbance profiles of degradation products overlap, an HPLC method will be developed to quantify the amount of HC released at a specific time point.

It is expected that the more hydrophilic the linker molecule, the faster the drug release. The drug release profiles of hydrogels may be very different than the PAE alone due to the formulation into hydrogels as the polymers may no longer be surface eroding. Nonetheless, the systems described here should be advantageous over current systems as water is necessary to hydrolyze the anhydride and ester bonds before drug release. Additionally, copolymers of PAEs with different linkers may be used to obtain the desired drug release profiles.

### Variation of Exposure Time to UV Radiation will Lead to Physicochemical Changes within the Hydrogel Blends.

Studies have shown increased exposure time to UV radiation significantly enhances the mechanical integrity of a hydrogel. One of the common methods to achieve randomly crosslinked networks is photocrosslinking, which has the advantages of simpler processing and the absence of a potentially toxic crosslinking agent (Lopergolo, et al., Polymer 2003, 44, (20), 6217-6222; Zhang, et al., Journal of Polymer Science Part A: Polymer Chemistry 2000, 38, (13), 2392-2404). Irradiation of the blends may alter the drug release profile of the hydrogels.

Preparation of Hydrogel Blends.

Solvent Casting Blends: To study the effects of crosslinking on the mechanical properties of a hydrogel blend, 8a will be used as its drug release properties will likely be the intermediate compared to 8b and 8c. Once synthesized, 8a will be dissolved with PVP in a mutual solvent, cast onto Teflon plates, and dried under vacuum.

Varying UV Radiation Exposure Time: The mechanical properties are dependent upon radiation exposure time. Sample films will be subjected to UV radiation (λₑₘ=254 nm) over one hour exposure time at fifteen minute intervals via techniques outlined by Lopérgolo et al. to induce chemical crosslinking (Polymer 2003, 44, (20), 6217-6222). This will help determine correlations between UV exposure time and mechanical properties to allow optimization of mechanical properties.

Characterization of Hydrogel Blends. The hydrogel blends will be characterized using DSC, swelling ratio, IR and SEM, as described above. Additionally, the following items will also be examined.

Mechanical Properties: Parallel plate rheometry will be carried out on the samples previously swelled in PBS for 24 hours. A force (depending on the strength of the hydrogel) will be exerted on the samples to determine the point where the sample's interactions are increasingly stretched until broken. This will determine the type of application the hydrogel can be useful for. If a broad range of mechanical properties are not found with 7:3 PVP:PAE, other ratios will be tested.

IR: The hydrogel blends will be characterized by IR as described above. In addition, the IR spectrum for crosslinked PVP will result in a peak at 1660 cm⁻¹. The absorptions from the trans ethylene group (1600 and 1320 cm⁻¹) will likely decrease with cleavage of the unsaturated bond as UV irradiation time increases.

Solubility: A decrease in solubility upon UV radiation will be indicative of crosslinking within the blend. This will be assessed in a variety of solvents including water, methanol, dimethylformamide, and toluene.

Gel Content: After UV radiation exposure, the gel content will be quantified. The irradiated films (IF) will be washed with chloroform to dissolve and remove unreacted polymer. The insoluble gel (IG) will be dried under vacuum and weighed. The percent gel content will be calculated by: [w_{IG}/w_{IF}]*100, where W_{IG} is the weight of the insoluble gel and W_{IF} is the weight of the films immediately following UV exposure.

In Vitro Hydrolytic Degradation of Hydrogel Blends. The in vitro drug release from the hydrogel blends will be examined via in vitro studies in PBS under simulated physiological conditions, UV and HPLC, as outlined above.

It is expected that as UV irradiation time increases, the physicochemical properties will be altered. Materials exhibiting greater mechanical strengths over other hydrogels can be achieved. A decrease in solubility is also expected as there will be less water accessibility, thus leading to longer drug release. Furthermore, drug release profiles may be different as the degradation profile usually observed by polyanhydrides may not occur for the hydrogel blends. An external crosslinking agent to provide crosslinking may need to be used; however, based upon previous work, the need for a photoinitiator seems unlikely (Nagata, M. et al., Polymer 2004, 45, (1), 87-93; Nagata, et al., Macromolecular Bioscience 2003, 3, (8), 412-419).

High drug loading can be obtained by creating hydrogels composed solely of PAEs.

The success of PVP for its physical integrity and commercial use as a hydrogel is beneficial for the aforementioned blends. However, creating blends lowers the overall drug loading capabilities of the hydrogels. If the concepts outlined above were used for PAEs alone (no blends), higher drug loading would be achieved.

Hydrogel Preparation.

Adding additional crosslinking sites: Use of linker molecules with photoreactive double bonds would be beneficial as PVP will no longer be present and therefore additional crosslinking sites (in addition to the double bond in the HC backbone) may be necessary. Also, this methodology of using unsaturated linker molecules can be adapted and used for bioactive-based PAEs that do not have a photoreactive double bond. This can be achieved by using acyl chlorides such as itaconyl chloride or fumaryl chlorides. A synthetic outline for PAEs with these linkers is shown in Scheme 5.

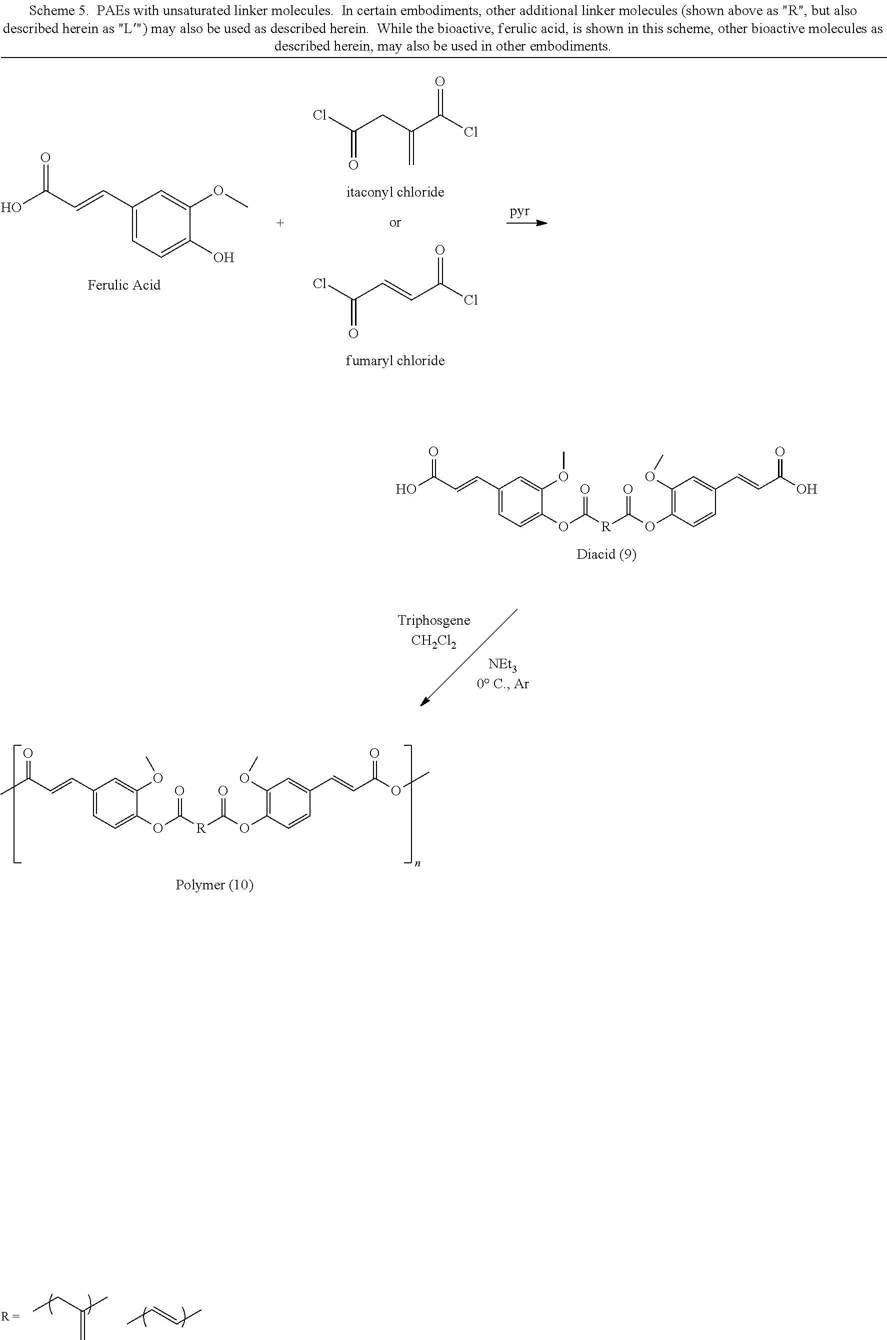

Characterization methods for (9) and (10), as outlined above, will be performed. Specifically, (9) and (10) will be characterized using nuclear magnetic resonance (NMR) (¹H and ¹³C) and Fourier transform infrared (FTIR) spectroscopies. The melting point (Tₘ) and glass transition temperature (T_{g}) will be obtained via differential scanning calorimetry (DSC), decomposition temperature using thermogravimetric analysis, and molecular weight by gel permeation chromatography methods.

Solvent Casting: Synthesis of 10 according the above schematic will be performed. Once synthesized, the PAE will be dissolved with an organic solvent and cast into films.

Varying UV Radiation Exposure Time: The UV radiation exposure time will be varied as described above.

Characterization of Hydrogel

DSC: To determine the thermal properties of the polymer, the T_{g} of the PAE film will be obtained.

Additional characterization will be performed as described above, including the swelling ratio, IR, SEM, mechanical properties, solubility and gel content.

In Vitro Hydrolytic Degradation of Hydrogel. The in vitro hydrolytic degradation and drug release will be examined as described above, including by the previously described in vitro studies in PBS under simulated physiological conditions, UV and HPLC.

The formation of a crosslinked PAE network is expected. More hydrophilic copolymers may be prepared if the system is determined to be too hydrophobic. Steric hindrance may be a potential limitation with bioactives with substituent groups as the proximity of the double bond to crosslink may not be feasible. Because an increased exposure time may result in a more mechanically stable hydrogel, UV irradiation time may be altered accordingly to ensure that the majority of the PAE is not crosslinked allowing ferulic acid to maintain bioactivity when hydrolytically released.

The experiments described above may be used to evaluate the controllability of the drug release profile of HC-based PAEs formulated into hydrogels. Additionally, changes in exposure to UV radiation may be used to independently control the mechanical properties of the hydrogel. With this, current limitations of using hydrogels for drug delivery can be overcome.

### EXAMPLE 5

Chronic wounds, including burns, skin ulcers and bed sores, are associated with high morbidity and mortality rates and affect ˜5.7 million patients, resulting in an estimated cost of $20 billion annually in the United States. Complications from chronic wounds can result from improper healing (hypertrophic scarring), dehydration and infection. Accordingly, improved wound dressings are needed.

Accordingly, as described herein, are hydrogels that can reduce pain, remove exudate, provide protection as a physical barrier, release bioactives, provide gas exchange, have an ideal humidity and/or can be easily applied and removed. In certain emboidments, the hydrogels comprise PVP and PAEs. In certain embodiments, the PAEs comprise salicylic acid, which is a non-steroidal anti-inflammatory drug, as well as an analgesic and antiseptic.

High water content and large pore size of hydrogels often results in rapid drug release and burst release of SA may cause toxicity issues. Accordingly, as described herein, are hydrogels that comprise PAEs with chemically incorporated SA in the PAE backbone. The addition of chemically incorporated SA, as opposed to physical incorporation, into a poly(anhydride-ester) (PAE) backbone would prevent burst release and enable high drug loading (up to 75%). These polymers are completely biodegradable, proven to exhibit controlled, sustained drug release profiles and have been established in vivo. A schematic illustrating SA release upon PAE hydrolysis is shown below (Scheme 6). SA release may be tuned by changing linker structure. For example, oxygen containing linkers can result in 100% drug release within days, linear aliphatic linkers can result in 100% drug release in weeks and branched aliphatic within months.

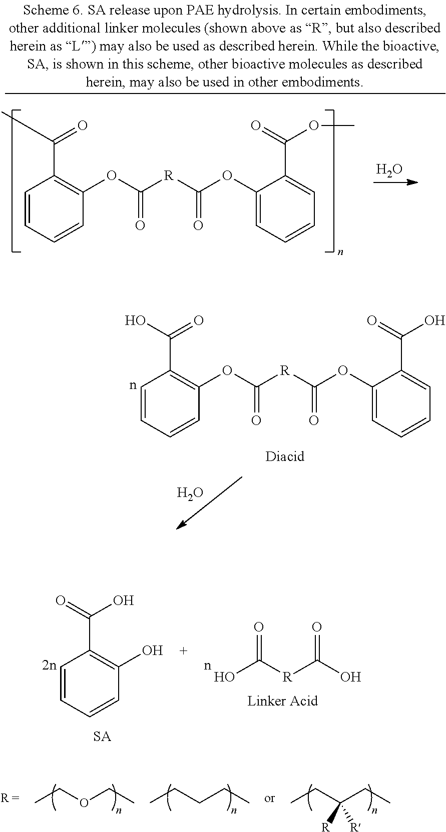

Two salicylate-based poly(anhydride-esters) (PAEs) of differing hydrophilicity (aliphatic vs. heteroatom “linkers”) were prepared and blended with PVP at various ratios. To elucidate the effect of linker structure on physical properties of the blends, they were cast into films and hydrated to form physical hydrogels. In general, the blends with the PAE aliphatic linker had higher swelling values than their counterparts.

PVP:SA (diglycolic) PAE Hydrogels

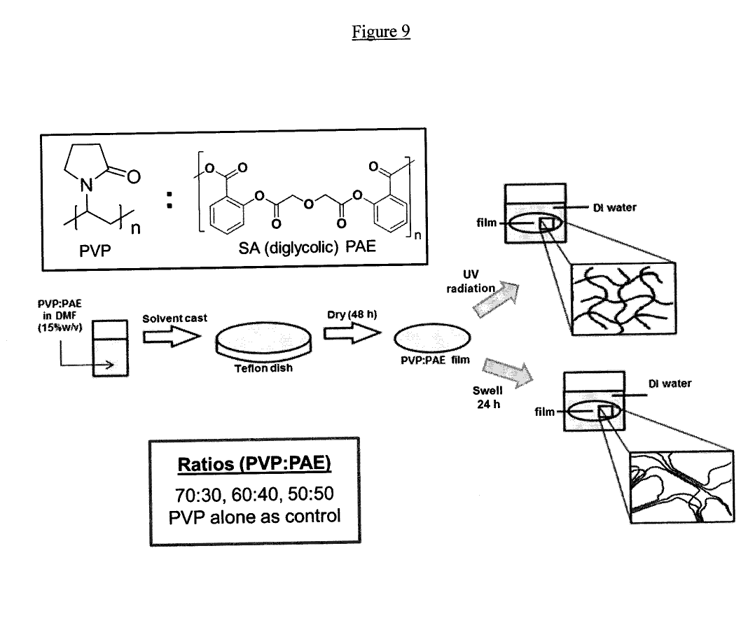

SA (diglycolic) PAE releases SA within the range useful for wound dressings (2-7 days). Formulation of these SA (diglycolic) PAEs into hydrogels can be obtained by blending with PVP as shown in FIG. 9. These PVP:PAE hydrogels were generated with the following ratios: 70:30, 60:40 and 50:50; PVP alone was used as a control. The PVP:PAE hydrogels used in the following experiments were not subjected to UV radiation.

Swelling of Physical Hydrogels

The films swelled in DI water for 24 hours at room temperature. The 100% PVP control hydrogels dissolved upon hydration. Swelling values (Q) were calculated using the following equation: Q=(Wₛ−W_{d})/W_{d}, wherein Wₛ and W_{d} are the weight of swollen and dried hydrogels, respectively. The calculated swelling values for each PVP:PAE hydrogel ratio are shown in Table 6.

TABLE 6

| PVP:SA(diglycolic)PAE Ratio | Swelling Value (g) |
|---|---|
| 70:30 | 0.6 ± 0.0 |
| 60:40 | 2.0 ± 0.7 |
| 50:50 | 1.7 ± 0.2 |

Low swelling values were obtained after swelling for 24 hours in DI water at room temperature (Q for chemically crosslinked PVP was ˜20). Additionally, no swelling trend was observed and the blended gels were fragile.

In Vitro Release Studies

Release studies were performed using physiological conditions (phosphate buffered saline (PBS) at pH 7.4 and 37° C.) and at 60 rpm. Every 24 hours an aliquot of media was removed and replaced with fresh PBS. Spent media was analyzed by UV-vis spectrophotometry at λ=303 nm; SA absorbs at this wavelength whereas other degradation products do not. The degradation of the SA(diglycolic) PAE is shown below in Scheme 7.

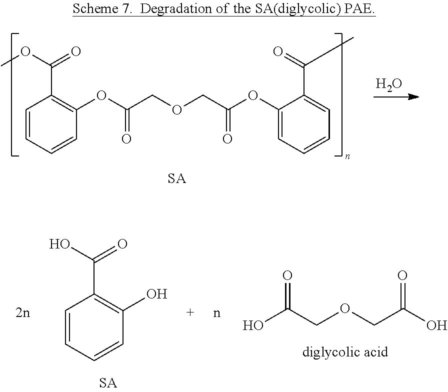

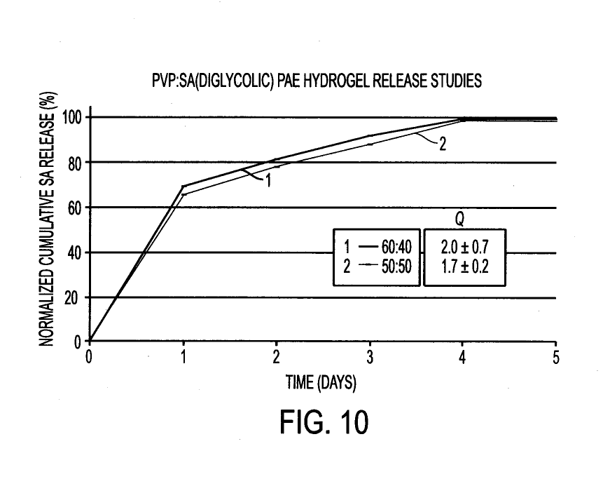

Dissolution occurred for the 70:30 samples, whereas for the other ratios the chemically incorporated SA was released over 4 days. As shown in FIG. 10, the percent of SA release correlated with the swelling values.
PVP:SA (adipic) PAE Hydrogels

PVP:SA (adipic) PAEs hydrogels, as shown below, can be similarly obtained as described above and as shown in FIG. 9. The PVP:SA (adipic) PAEs hydrogels used in the following experiments were not subjected to UV radiation. These PVP:PAE hydrogels were generated with the following ratios: 70:30, 60:40 and 50:50.

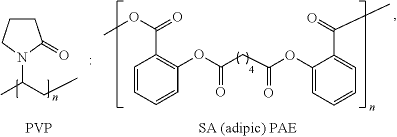

Swelling of Physical Hydrogels

Films swelled in DI water for 24 hours at room temperature. The swelling values (Q) were calculated as described above and compared to the values obtained for the diglycolic-containing PAE blends (Table 7).

TABLE 7

| | PVP:SA (adipic) PAE | PVP:SA (diglycolic) |
|---|---|---|
| Ratio | Swelling Value (Q) | PAE Swelling Value (Q) |
| 70:30 | 14.2 ± 1.4 | 0.6 ± 0.0 |
| 60:40 | 7.2 ± 0.8 | 2.0 ± 0.7 |
| 50:50 | 4.9 ± 0.1 | 1.7 ± 0.2 |

Higher swelling values were obtained for adipic-containing PAE compared to diglycolic-containing PAEs and Q increased as PVP content increased.

Scanning Electron Microscropy (SEM)

To observe the porosity of the hydrogels, samples were swelled in DI water for 6 and 24 hours and analyzed by SEM. Porosity is important for gas exchange for proper healing. More PVP resulted in greater porosity and greater porosity was observed at increased time points. The 7:3 PVP/PAE ratio had pore sizes of about <10 μ.m. Additionally, SEM images indicate that degradation may be taking place at the 24 hour time point. Further studies may be performed to elucidate swelling kinetics (e.g., additional ratios and swelling time points).

In Vitro Release Studies

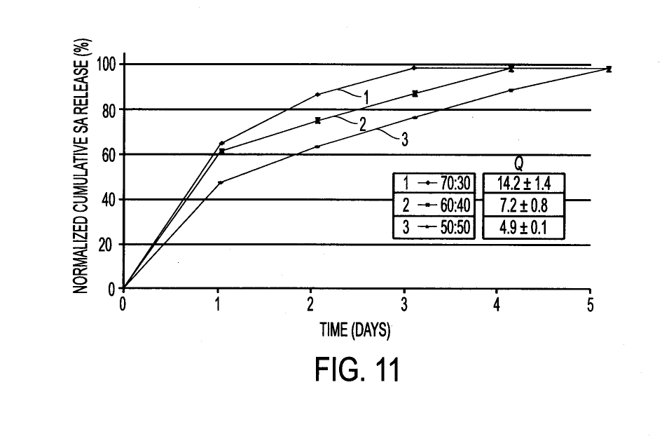

Release studies were performed using the same conditions as described above for the diglycolic-containing blends. Normalized cumulative SA release (%) for the three ratios is shown in FIG. 11. The chemically incorporated SA sustained release over 4-5 days. Additionally, the SA release followed the swelling trend: 70:30>60:40>50:50.

As a control, PVP:poly(anhydride-ether) blends with physically incorporated SA (Scheme 8A) were generated. The hydrolysis of a poly(anhydride-ether) is shown below in Scheme 8B.

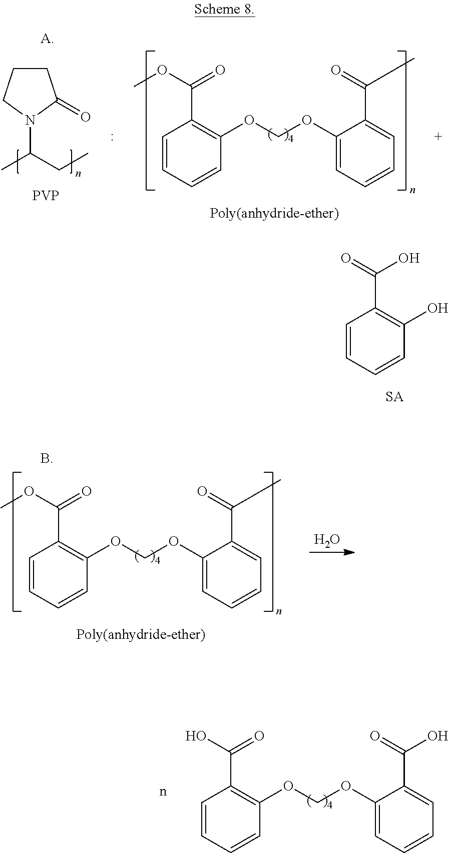

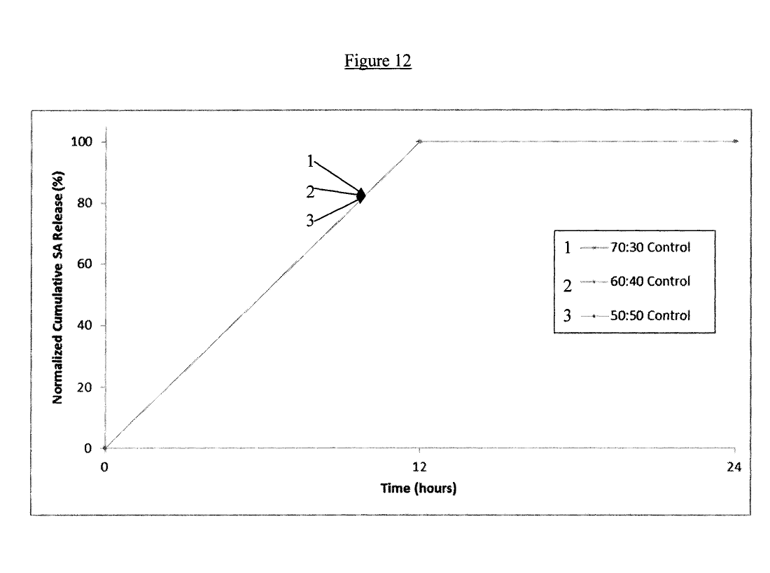

As expected, the physically mixed SA was released in less than 1 day for all samples (FIG. 12), demonstrating chemically incorporated SA is advantageous over physical incorporation.

### PVP:SA (Adipic) PAE & PVP:SA (Diglycolic) PAE Hydrogels

Cytotoxicity Studies

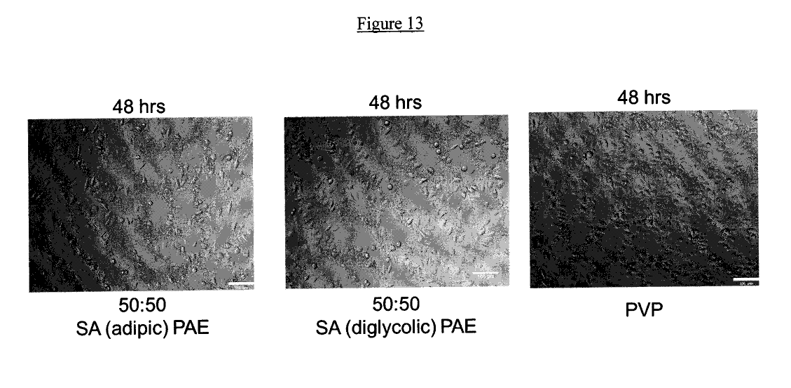

Polymer films were sterilized for 15 minutes via UV radiation. The L929 mouse fibroblast cell line was used for the cytotoxicity studies. The cells were plated in 96 well plates. Polymer dissolved in DMSO was added to cell media at polymer concentrations of 0.1 mg/mL and 0.01 mg/mL; DMSO used as a control. Cell morphology was evaluated over 3 days and elongated morphology was observed (FIG. 13).

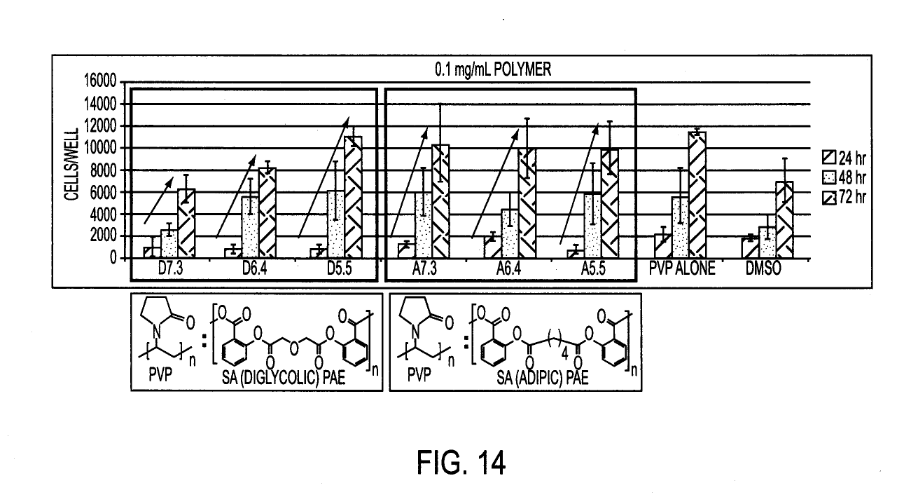

Additionally, the MTS assay was performed to measure cell viability over three days. High and low concentrations exhibited similar trends. This assay similarly showed an increase in cell viability over time for all samples (FIG. 14). No statistical differences were observed between the samples and the DMSO control.

Other Studies

Mechanical studies can be performed to determined tensile strength and elongation at break. Based on other research using other dressings, possible desired tensile strength and elongation at break values may be approximately about 13 MPa and about 40%, respectively. Additionally, other studies may also be performed to further analyze the hydrogels, including: infrared spectroscopy to elucidate interactions taking place to form the gel; in vitro cytokine studies to investigate the anti-inflammatory activity of the hydrogels and to confirm negligible skin irritation; and skin permeation testing. In addition to SA, other bioactive-based PAEs that may be beneficial for topical applications may also be used and could be examined using the experiments described herein or known in the art.

As described herein polymer blends have been formulated into a hydrogel with good handling capabilities, swelling (appropriate Q values) and porosity for gas exchange. Blending SA-based PAEs with PVP to formulate into hydrogels allows for both sustained SA release (over 4-5 days) and a soft materials provided by the PVP, which is beneficial for topical applications. Additionally, these polymer blends were shown to be cytocompatible, and therefore, would be appropriate for use as active wound dressings.

All documents cited herein are incorporated by reference. While certain embodiments of invention are described, and many details have been set forth for purposes of illustration, certain of the details can be varied without departing from the basic principles of the invention.

The use of the terms “a” and “an” and “the” and similar terms in the context of describing embodiments of invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. In addition to the order detailed herein, the methods described herein can be performed in any suitable order unless otherwise indicated herein or otherwise clearly contradicted by context. The use of any and all examples, or exemplary language (e.g., “such as”) provided herein, is intended merely to better illuminate embodiments of invention and does not necessarily impose a limitation on the scope of the invention unless otherwise specifically recited in the claims. No language in the specification should be construed as indicating that any non-claimed element is essential to the practice of the invention.

What is claimed is:

1. A hydrogel comprising (a) a poly(anhydride-ester) comprising a polymer backbone and having a group in the polymer backbone that will yield a bioactive molecule upon hydrolysis of the polymer backbone; and (b) a hydrophilic polymer that is crosslinked with the poly(anhydride-ester).

2. The hydrogel of claim 1, wherein the bioactive molecule is an antimicrobial, anti-inflammatory, antioxidant or analgesic.

3. The hydrogel of claim 1, wherein the bioactive molecule is a hydroxycinnamate or a salicylate.

4. The hydrogel of claim 3, wherein the hydroxycinnamate is selected from ferulic acid, sinapic acid and p-coumaric acid.

5. The hydrogel of claim 3, wherein the salicylate is salicylic acid.

6. The hydrogel of claim 1, wherein the poly(anhydride-ester) comprises one or more units of formula (I) in the backbone:
—C(═O)X¹-L-X¹C(═O)—O— (I)
wherein
each X¹ is independently a group that will provide a biologically active compound upon hydrolysis of the polymer; and
L is independently a linker molecule.

7. The hydrogel of claim 6, wherein each linker molecule is selected from a branched aliphatic, linear aliphatic, and oxygen-containing linker molecule.

8. The hydrogel of claim 7, wherein the branched aliphatic linker molecule is derivable from diethylmalonyl chloride.

9. The hydrogel of claim 7, wherein the linear aliphatic linker molecule is derivable from adipoyl chloride.

10. The hydrogel of claim 7, wherein the oxygen-containing linker molecule is derivable from diglycolyl chloride.

11. The hydrogel of claim 1, wherein in the poly(anhydride-ester) is: 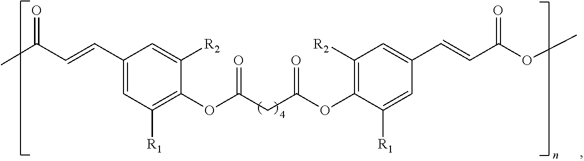
wherein R₁ is OCH₃ and R₂ is H; R₁ is OCH₃ and R₂ is OCH₃; or R₁ is H and R₂ is H; and wherein n is 2 or more.

12. The hydrogel of claim 1, wherein in the poly(anhydride-ester) is: 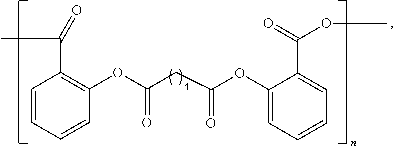
wherein n is 2 or more.

13. The hydrogel of claim 1, wherein the ratio of the poly(anhydride-ester) to the hydrophilic polymer ranges between about 1:9 to about 1:1.

14. The hydrogel of claim 1, wherein the hydrophilic polymer comprises poly(N-vinyl-2-pyrrolidone), polyvinylpolypyrrolidone, poly(vinyl alcohol), polyurethane or poly(ethylene oxide).

15. The hydrogel of claim 14, wherein the hydrophilic polymer is poly(N-vinyl-2-pyrrolidone).

16. The hydrogel of claim 1, wherein the poly(anhydride-ester) is physically crosslinked with the hydrophilic polymer through hydrophobic interactions.

17. A method of making a hydrogel as described in claim 1, comprising solvent casting (a) a poly(anhydride-ester) comprising a polymer backbone and having a group in the polymer backbone that will yield a bioactive molecule upon hydrolysis of the polymer backbone; and (b) a hydrophilic polymer; under conditions to provide a hydrogel.

18. The method of claim 17, further comprising cross-linking the poly(anhydride-ester) with the hydrophilic polymer using ultraviolet radiation, gamma radiation or an external cross-linking agent.

19. A method for promoting wound healing in a mammal, comprising contacting a hydrogel as described in claim 1 with a wound of the mammal.

20. A method of therapeutically treating the skin of a mammal, comprising contacting a hydrogel as described in claim 1 with the skin of the mammal.
